(19) United States
(12) Patent Application Publication
(10) Pub. No.: US 2010/0113570 A1
(43) Pub. Date: May 6, 2010

(54) PREVENTION AND TREATMENT OF POST-OPERATIVE COGNITIVE DYSFUNCTION

(75) Inventors: Zhongcong Xie, Andover, MA (US); Rudolph E. Tanzi, Hull, MA (US)

Correspondence Address: WOLF GREENFIELD & SACKS, P.C., 600 ATLANTIC AVENUE, BOSTON, MA, 02210-2206, US

(73) Assignees: The General Hospital Corporation, Boston, MA (US); D/B/A MASSACHUSETTS GENERAL HISPITAL, BOSTON, MA (US)

(21) Appl. No.: 12/428,597

(22) Filed: Apr. 23, 2009

Related U.S. Application Data
(60) Provisional application No. 61/047,308, filed on Apr. 23, 2008.

(51) Int. Cl. A61K 31/08; A61K 31/02; A61K 31/13; A61P 25/00; A61K 31/7088

(52) U.S. Cl. 514/44 R; 514/722; 514/743; 514/740

(57) ABSTRACT

Methods for the use of uncompetitive NMDA receptor antagonist(s) in inhalational anesthesia are provided herein.

### RELATED APPLICATIONS

This application claims the benefit under 35 U.S.C. §119(e) of U.S. provisional application Ser. No. 61/047,308 filed on Apr. 23, 2008, the entire disclosure of which is incorporated herein by reference.

### FEDERALLY SPONSORED RESEARCH

Research leading to certain aspects of the invention(s) disclosed herein was supported at least in part by National Institutes of Health (NIH) Grants NS048140, AG014713, MH60009, GM077057, and AG20253. The United States Government has certain rights to the invention.

### BACKGROUND OF THE INVENTION

Increasing evidence indicates that caspase activation and apoptosis are associated with a variety of neurodegenerative disorders, including Alzheimer's Disease, as well as with cognitive dysfunction. We reported previously that commonly used inhalational anesthetics, for example halogenated ethers, can induce apoptosis, alter processing of the amyloid precursor protein (APP), and increase amyloid-β protein (Aβ) generation. Further, postoperative cognitive dysfunction (POCD) is often observed in subjects after inhalational anesthesia.

### SUMMARY OF THE INVENTION

Commonly used inhalational anesthetics can increase the risk of developing Alzheimer's Disease (AD) in subjects undergoing anesthesia and are often associated with postoperative cognitive dysfunction (POCD), characterized by an impairment, temporary or permanent, of cognitive function.

The mechanism by which inhalational anesthetic agents, for example halogenated ethers, such as isoflurane, enflurane, halothane, sevoflurane, and desflurane, increase the risk of Alzheimer's Disease and cause POCD is poorly understood. It has been suggested that caspase activation and consequent induction of apoptosis, as well as induction of alterations in processing of the amyloid precursor protein (APP), and increase of amyloid-β protein (Aβ) generation in the brain may play key roles in these undesired effects.

Understanding the underlying molecular mechanism is a prerequisite for developing improved methods of anesthesia that are not burdened, or associated to a lesser extent, with such undesired side effects. We report here insights into the molecular mechanism causing the detrimental side effects of elevated AD risk and POCD of halogenated ether anesthetic agents. Further, improved methods of anesthesia that avoid or ameliorate these detrimental effects by administering an uncompetitive NMDA receptor antagonist in temporal proximity to a clinical intervention known to increase the risk of developing AD or to cause OPCD, for example an inhalational anesthesia, are provided herein.

According to one aspect of the invention, methods are provided that include administering an uncompetitive N-methyl D-aspartate (NMDA) receptor antagonist to a subject in temporal proximity to administering a halogenated ether anesthetic to the subject. In some embodiments, the halogenated ether anesthetic is selected from the group consisting of isoflurane, enflurane, halothane, sevoflurane and desflurane. In certain embodiments, the halogenated ether anesthetic is isoflurane. In some embodiments, the uncompetitive NMDA receptor antagonist is memantine or a pharmaceutically acceptable salt thereof.

In some embodiments, the uncompetitive N-methyl D-aspartate (NMDA) receptor antagonist and the halogenated ether anesthetic are administered to the subject within one hour of each other. In certain embodiments, the uncompetitive N-methyl D-aspartate (NMDA) receptor antagonist is administered to the subject prior to the halogenated ether anesthetic.

In some embodiments, the uncompetitive NMDA receptor antagonist is administered in an amount sufficient to decrease a level of amyloid-β or to prevent an increase in a level of amyloid-β in a cell of said subject and/or in an amount sufficient to reduce or prevent apoptosis in a cell in said subject and/or in an amount sufficient to prevent or inhibit a cognitive impairment or to decrease the level of a cognitive impairment in said subject.

In some embodiments, the subject is human.

According to another aspect of the invention, methods for inhibiting postoperative cognitive dysfunction (POCD) induced by administration of a halogenated ether anesthetic to a subject are provided. The methods include administering an uncompetitive N-methyl D-aspartate (NMDA) receptor antagonist to a human subject in temporal proximity to administering the halogenated ether anesthetic to the subject. In some embodiments, the halogenated ether anesthetic is selected from the group consisting of isoflurane, enflurane, halothane, sevoflurane and desflurane. In certain embodiments, the halogenated ether anesthetic is isoflurane. In some embodiments, the uncompetitive NMDA receptor antagonist is memantine or a pharmaceutically acceptable salt thereof.

In some embodiments, the uncompetitive N-methyl D-aspartate (NMDA) receptor antagonist and the halogenated ether anesthetic are administered to the subject within one hour of each other. In certain embodiments, the uncompetitive N-methyl D-aspartate (NMDA) receptor antagonist is administered to the subject prior to the halogenated ether anesthetic.

In some embodiments, the uncompetitive NMDA receptor antagonist is administered in an amount sufficient to decrease a level of amyloid-β or to prevent an increase in a level of amyloid-β in a cell of said subject and/or in an amount sufficient to reduce or prevent apoptosis in a cell in said subject and/or in an amount sufficient to prevent or inhibit a cognitive impairment or to decrease the level of a cognitive impairment in said subject.

In some embodiments, the subject is human.

According to yet another aspect of the invention, methods are provided that include administering an inhibitory nucleic acid molecule directed against IP3 receptor or SERCA1 and/or a chelating agent to a subject in temporal proximity to administering a halogenated ether anesthetic to the subject. In some embodiments, the halogenated ether anesthetic is selected from the group consisting of isoflurane, enflurane, halothane, sevoflurane and desflurane. In some embodiments, the inhibitory nucleic acid molecule and/or the chelating agent, and the halogenated ether anesthetic are administered to the subject within one hour of each other. In some embodiments, the subject is human.

In some embodiments of the foregoing methods, the subject is not diagnosed or indicated to have Alzheimer's Disease, while in other embodiments the subject is diagnosed or indicated to have Alzheimer's Disease. In some embodiments, the subject is not diagnosed or indicated to have an impairment in cognition, while in other embodiments the subject is diagnosed or indicated to have an impairment in cognition.

Kits useful in carrying out the methods described herein also are provided. Such kits contain one or more, typically two or more containers of the components employed in the methods, and optionally contain instructions for the use of the components in carrying out the methods described herein.

Other advantages, features, and uses of the invention will become apparent from the following detailed description of non-limiting embodiments of the invention when considered in conjunction with the accompanying drawings. In cases where the present specification and a document incorporated by reference include conflicting disclosure, the present specification shall control.

### BRIEF DESCRIPTION OF THE DRAWINGS

FIG. 1. Memantine attenuates isoflurane-induced caspase-3 activation and apoptosis in H4-APP cells. A, Treatment with 2% isoflurane for 6 h (lane 4) induces caspase-3 cleavage (activation) compared with control conditions (lane 1) in H4-APP cells. Treatment with 4 μM memantine alone does not cause caspase-3 activation (lanes 2 and 3) compared with control condition (lane 1). However, treatment with isoflurane plus memantine (lanes 5 and 6) causes a lesser degree of caspase-3 activation compared with isoflurane treatment alone (lane 4). There is no significant difference in amounts of β-actin in all of the above treatments. B, The 2% isoflurane treatment (black) increases caspase-3 activation compared with control conditions (white),normalized to β-actin levels. Memantine treatment alone (gray) does not induce caspase-3 activation compared with control condition (white); however, memantine treatment (lined) attenuates the isoflurane-induced (black) caspase-3 activation, normalized to β-actin levels. C, Isoflurane treatment (2%) (black) decreases cell viability compared with control conditions (white). Memantine treatment alone (gray) does not affect cell viability compared with control conditions (white); however, memantine treatment (lined) attenuates the isoflurane-induced reduction in cell viability (black). D, Isoflurane treatment (2%) (black) induces apoptosis compared with control conditions (white). Memantine treatment alone (gray) does not induce apoptosis compared with control conditions (white); however, memantine treatment (lined) attenuates isoflurane-induced apoptosis (black). E, H4-APP cells contain NR1. Western blot analysis illustrates that anti-NR1 antibody (lanes 1-3) detects NR1 in H4-APP cells, whereas NR1 peptide (lanes 4-6) prevents anti-NR1 antibody from detecting NR1 in H4-APP cells. These results suggest that there is NR1 in H4-APP cells. Data are means±SD (n=3-9 for each experimental group). A t test is used to compare the difference between control condition and the isoflurane or memantine treatment. The asterisk indicates the difference between isoflurane and control condition on caspase-3 activation (*p=0.015), cell viability (*p=0.001), and apoptosis (*p=0.001); the difference between saline and memantine on isoflurane-induced caspase-3 activation (#p=0.013), cell viability (#p=0.037), and apoptosis (#p=0.001) is indicated.

FIG. 2. Isoflurane-induced elevation of cytosolic calcium levels can be inhibited by memantine in H4-APP cells. A, Isoflurane elevates cytosolic calcium levels in H4-APP cells. Isoflurane (1 mM; or 3.5%) was given to H4-APP cells at time 0. Cytosolic calcium levels were elevated after isoflurane treatment in H4-APP cells. B, Memantine inhibits isoflurane-induced elevation of cytosolic calcium levels in H4-APP cells. H4-APP cells were pretreated with 4 μM memantine for 5 mM and then exposed to 1 mM (or 3.5%) isoflurane at time 0. Note that cytosolic calcium levels were not elevated by isoflurane in the memantine-pretreated H4-APP cells. These are representative tracings of two independent experiments.

FIG. 3. Memantine attenuates isoflurane-induced caspase-3 activation in naive mice. A, Isoflurane treatment (lane 3) induces caspase-3 cleavage (activation) compared with control conditions (lane 1) in naive mice. Treatment with memantine alone does not cause caspase-3activation (lane 2) compared with control condition (lane 1). However, treatment with isoflurane plus memantine (lanes 4) causes a reduction in caspase-3 activation compared with isoflurane treatment alone (lane 3). There is no significant difference in the amounts of β-actin in all of the treatments. The Western blot, which shows the caspase-3 fragment (17 kDa) only, is the same Western blot with longer exposure time during the film development. B, Isoflurane treatment (black) increases caspase-3 activation compared with control conditions (white), normalized to β-actin levels, in naive mice. Memantine treatment alone (gray) does not induce caspase-3 activation compared with control condition (white); however, memantine treatment(lined) attenuates the isoflurane-induced (black) caspase-3 activation, normalized to β-actin levels. Data are means±SD (n=4 for each experimental group). A t test is used to compare the difference between control condition and the isoflurane or memantine treatment. *p=0.034, difference between isoflurane and control condition on caspase-3 activation; #p =0.020, difference between saline and memantine on isoflurane-induced caspase-3 activation.

FIG. 4. RNAi knockdown of NR1 reduces isoflurane-induced caspase-3 activation in H4-APP cells. A, NR1 siRNA (lanes 3 and4) reduces protein levels of NR1 compared with control siRNA treatment (lanes 1 and 2). There is no significant difference in amounts of β-actin in control or NR1 siRNA-treated cells. B, Quantitation of the Western blots shows that NR1 siRNA treatment(black) reduces protein levels of NR1 compared with control siRNA treatment (white). A t test is used to compare difference between control siRNA and NR1 siRNA treatment in reducing NR1 protein levels (*p=0.0439). C, Treatment with control siRNA plus isoflurane (lanes 5 and 6) induces caspase-3 activation compared with control siRNA plus control condition (lanes 1 and 2). NR1 siRNA alone (lanes 3 and 4) does not induce caspase-3 activation. However, treatment with NR1 siRNA plus isoflurane (lanes 7 and 8) induces a lesser degree of caspase-3 activation compared with a treatment with control siRNA plus isoflurane (lanes 5 and6). The Western blot, which shows the caspase-3 fragment (17 kDa) only, is the same Western blot with longer exposure time during the film development. D, Quantification of the Western blot shows that control condition plus NR1 siRNA treatment (gray) does not induce caspase-3 activation compared with control condition plus control siRNA treatment (white). The treatment with isoflurane plus control siRNA (black) induces caspase-3 activation compared with control condition plus control siRNA treatment(white). NR1 siRNA treatment (lined) attenuates the isoflurane-induced (black) caspase-3 activation. Data are means±SD (n=3-6 for each experimental group). A t test is used to compare the difference. *p=0.041, difference between isoflurane and control condition on caspase-3 activation; #p=0.037, difference between control siRNA and NR1 siRNA on isoflurane-induced caspase-3 activation.

FIG. 5. EDTA, low calcium, and BAPTA attenuate isoflurane-induced caspase-3 activation in H4-APP cells. A, Treatment with isoflurane plus low-calcium condition (lanes 3 and 4) yields a reduction in caspase-3 cleavage (activation) compared with isoflurane treatment alone (lanes 1 and 2) in H4-APP cells. Treatment with isoflurane plus low-calcium condition and EDTA (lanes 5 and 6) causes an even lesser degree of caspase-3 activation compared with either isoflurane treatment alone (lanes 1 and 2) or the treatment of isoflurane plus low-calcium condition (lanes 3 and 4). There is no significant difference in the amounts of β-actin in all of the above treatments in H4-APP cells. The Western blot, which shows the caspase-3 fragment (17 kDa) only, is the same Western blot with longer exposure time during the film development. B, Caspase-3 activation assessed by quantifying the ratio of caspase-3 fragment to FL-caspase-3 in the Western blots. The treatment with isoflurane and low-calcium condition (gray)induces a reduction in caspase-3 activation compared with isoflurane treatment alone (white). The treatment with isoflurane plus low-calcium condition and EDTA (black) triggers an even lesser degree of caspase-3 activation compared with either isoflurane treatment alone (white) or the treatment with isoflurane plus low-calcium condition (gray). C, Treatment with DMSO plus isoflurane (lanes 5 and 6) induces caspase-3 activation compared with DMSO plus control condition (lanes 1 and 2). BAPTA alone(lanes 3 and 4) does not induce caspase-3 activation. However, treatment with BAPTA plus isoflurane (lanes 7 and 8) induces a lesser degree of caspase-3 activation compared with a treatment with DMSO plus isoflurane (lanes 5 and 6). D, Quantification of the Western blot shows that control condition plus BAPTA treatment (gray) dose not induce caspase-3 activation compared with control condition plus DMSO treatment (white). The treatment with isoflurane plus DMSO (black) induces caspase-3 activation compared with control condition plus DMSO treatment (white). BAPTA treatment (lined) attenuates the isoflurane-induced(black) caspase-3 activation. Data are means±SD (n=3-6 for each experimental group). A t test is used to compare the difference. *p=0.046, difference between isoflurane plus low-calcium condition and isoflurane plus normal calcium condition; *p=0.015, difference between isoflurane plus normal calcium condition and isoflurane plus low-calcium condition and EDTA; #p=0.029, difference between isoflurane plus low-calcium condition and isoflurane plus low-calcium condition and EDTA; *p=0.0103, difference between isoflurane and control condition on caspase-3 activation; #p=0.045, difference between BAPTA and control treatment on isoflurane-induced caspase-3 activation.

FIG. 6. RNAi knockdown of 1P3 receptor reduces isoflurane-induced caspase-3 activation in H4-APP cells. A, 1P3 receptor siRNA (lanes 2, 3, 5, and 6) reduces the protein levels of IP3 receptor compared with control siRNA treatment (lanes 1 and 4). The treatment with control siRNA plus isoflurane (lane 1) induces caspase-3 activation compared with control siRNA plus control condition (lane 4). The treatment of IP3 receptor siRNA plus control condition (lanes Sand 6) does not induce caspase-3 activation compared with control siRNA plus control condition(lane 4). However, the treatment with IP3 receptor siRNA plus isoflurane (lanes 2 and 3) yields a reduction in caspase-3 activation compared with the treatment with control siRNA plus isoflurane (lane 1). B, Quantitation of the Western blots shows that IP3 receptor siRNA treatment (black) reduces the protein levels of IP3 receptor compared with control siRNA treatment (white). A t test is used to compare the difference between control siRNA and IP3 receptor siRNA treatment in reducing IP3 receptor protein levels (*p=0.026). C, Control condition plus IP3siRNA treatment (gray) dose not induce caspase-3 activation compared with control condition plus control siRNA treatment (white). The treatment with either isoflurane plus control siRNA (black) or isoflurane plus IP3 receptor siRNA (lined) causes caspase-3 activation compared with control condition plus control siRNA treatment (white). However, IP3 receptor siRNA treatment (lined) attenuates the isoflurane-induced (black) caspase-3 activation. Data are means±SD (n=3-6 for each experimental group).At test is used to compare the difference. **p=0.001,**p=0.008, the difference between isoflurane and control condition on caspase-3 activation; #p=0.035, the difference between control siRNA and IP3 receptor siRNA on the isoflurane induced caspase-3 activation.

FIG. 7. RNAi knockdown of SERCA1 reduces isoflurane-induced caspase-3 activation in H4-APP cells. A, SERCA1 siRNA (lanes3 and 4) reduces protein levels of SERCA1 compared with control siRNA treatment (lanes 1 and 2). There is no significant difference in amounts of β-actin in control or SERCA1 siRNA-treated cells. B, Quantitation of the Western blots shows that SERCA1 siRNA treatment (black) reduces protein levels of SERCA1 compared with control siRNA treatment (white).At test is used to compare the difference between control siRNA and SERCA1 siRNA treatment in reducing SERCA1 protein levels (*p=0.021). C, Treatment with control siRNA plus isoflurane (lanes 4 and 5) induces caspase-3 activation compared with control siRNA plus control condition (lane 1). SERCA1 siRNA alone (lanes 2 and 3) does not induce caspase-3 activation. However, treatment with SERCA1 siRNA plus isoflurane (lanes 6 and 7) induces a reduction in caspase-3 activation compared with the treatment with control siRNA plus isoflurane (lanes 4 and 5). The Western blot, which shows the caspase-3 fragment (17 kDa) only, is the same Western blot with longer exposure time during the film development. D, Quantification of the Western blot shows that control condition plus SERCA1 siRNA treatment (gray) dose not induce caspase-3 activation compared with control condition plus control siRNA treatment (white). Treatment with isoflurane plus control siRNA (black) induces caspase-3 activation compared with control condition plus control siRNA treatment (white). SERCA1 siRNA treatment (lined) attenuates the isoflurane-induced caspase-3 activation (black). Data are means±SD (n=4 for each experimental group). A t test is used to compare the difference. **p=0.001, difference between isoflurane and control condition on caspase-3 activation; #p=0.012, difference between control siRNA and SERCA1 siRNA on isoflurane-induced caspase-3 activation.

### DETAILED DESCRIPTION OF THE INVENTION

Commonly used inhalational anesthetics can increase the risk of developing Alzheimer's Disease (AD) in subjects undergoing anesthesia and are often associated with postoperative cognitive dysfunction (POCD), characterized by an impairment, temporary or permanent, of cognitive function.

The mechanism by which inhalational anesthetic agents, for example halogenated ethers, such as isoflurane, enflurane, halothane, sevoflurane, and desflurane, increase the risk of Alzheimer's Disease and cause POCD is poorly understood. It has been suggested that caspase activation and consequent induction of apoptosis, as well as induction of alterations in processing of the amyloid precursor protein (APP), and increase of amyloid-β protein (Aβ) generation in the brain may play key roles in these undesired effects.

Understanding the underlying molecular mechanism is a prerequisite for developing improved methods of anesthesia that are not burdened, or associated to a lesser extent, with such undesired side effects. We report here insights into the molecular mechanism causing the detrimental side effects of elevated AD risk and POCD of halogenated ether anesthetic agents. Further, improved methods of anesthesia avoiding or ameliorating these detrimental effects by administering an uncompetitive NMDA receptor antagonist in temporal proximity to a clinical intervention known to increase the risk of developing AD or to cause OPCD, for example an inhalational anesthesia, are provided herein.

### Molecular Mechanism Underlying the Undesired Effects of Inhalational Anesthetic Agents

We set out to assess the molecular effects of extracellular calcium concentration on isoflurane-induced caspase-3 activation in H4human neuroglioma cells stably transfected to express human full-length APP (H4-APP cells). In addition, we tested effects of RNA interference (RNAi) silencing of IP3 receptor, NMDA receptor, and endoplasmic reticulum (ER) calcium pump, sacro-/ER calcium ATPase (SERCA1). Finally, we examined the effects of the NMDA receptor partial antagonist, memantine, in H4-APP cells and brain tissue of naive mice. EDTA (10 mM), BAPTA (10 μM), and RNAi silencing of IP3 receptor, NMDA receptor, or SERCA1 attenuated capase-3 activation. Memantine (4 μM) inhibited isoflurane-induced elevations in cytosolic calcium levels and attenuated isoflurane-induced caspase-3 activation, apoptosis, and cell viability. Memantine (20 mg/kg, i.p.) reduced isoflurane-induced caspase-3 activation in brain tissue of naive mice. These results suggest that disruption of calcium homeostasis underlies isoflurane-induced caspase activation and apoptosis. We also show for the first time that the NMDA receptor partial antagonist, memantine, can prevent isoflurane-induced caspase-3 activation and apoptosis in vivo and in vitro. These findings, indicating that isoflurane-induced caspase activation and apoptosis are dependent on cytosolic calcium levels, should facilitate the provision of safer anesthesia care, especially for Alzheimer's Disease and elderly patients.

### Alzheimer's Disease and Anesthesia

Cerebral deposition of amyloid-β protein (Aβ), derived from the amyloid precursor protein (APP), is a major pathological hallmark of Alzheimer's Disease (AD) (for review, see Selkoe, 2001; Tanzi and Bertram, 2005). Increasing evidence suggests a role for caspase activation and apoptosis in AD neuropathogenesis (Gervais et al., 1999; LeBlanc et al., 1999; Lu et al., 2000; Eckert et al.,2003; Gastard et al., 2003; Zhao et al., 2003; Hitomi et al., 2004; Takuma et al., 2004). Several studies showed the potential association of previous general anesthesia/surgery and risk of AD (Bohnen et al., 1994a,b; Muravchick and Smith, 1995). A previous study suggested that age of onset of AD was inversely related to anesthesia exposure before 50 years of age (Bohnen et al., 1994b). A recent study also reported that patients undergoing coronary artery bypass graft surgery under general anesthesia were at increased risk for AD compared with those having percutaneous transluminal coronary angioplasty under local anesthesia (Lee et al., 2005).

### Mechanisms for the Effect of Anesthetic Agents on AD Risk

Isoflurane, one of the most commonly used inhalation anesthetics, has been reported to enhance aggregation and cytotoxicity of Aβ (Eckenhoff et al., 2004) and induce caspase activation and apoptosis (Matsuoka et al., 2001; Jevtovic-Todorovic et al., 2003; Kvolik et al., 2005; Loop et al., 2005; Wei et al., 2005; Yon et al., 2005). Our recent studies have shown that a clinically relevant isoflurane treatment causes caspase-3 activation and apoptosis, decreases cell viability, affects APP processing, and increases AP generation in human neuroglioma cells stably transfected to express human APP (H4-APP cells) (Xie et al., 2006a,b, 2007).Moreover, isoflurane has been suggested to induce a vicious cycle of apoptosis and AP accumulation (Xie et al., 2007).The mechanism by which isoflurane induces caspase activation and apoptosis remains unclear. Wei et al. (2005, 2008) reported that dantrolene, an endoplasmic reticulum (ER) ryanodine receptor antagonist, and inositol 1,4,5-trisphosphate (IP3) receptor knock-out can inhibit isoflurane-induced apoptosis, suggesting that abnormal calcium release from ER after isoflurane treatment may cause apoptosis (Wei et al., 2005, 2008). Here, we used a variety of treatments in vitro and in vivo to alter calcium homeostasis and assessed the effects of these treatments on isoflurane-induced caspase-3 activation in H4-APP cells. The IP3 receptor, located in the ER membrane, regulates release of calcium from the ER to the cytoplasm (Berridge, 1993). Activation of IP3 receptors causes elevated cytosolic calcium levels, leading to cell death (Hanson et al., 2004; Lindholm et al., 2006). A recent study by Zhang et al. (2006) showed that the endoplasmic reticulum calcium pump, sacro-/ER calcium ATPase (SERCA1), is required for calcium release-activated channel activity, and RNA interference (RNAi) knockdown of SERCA1 inhibits calcium release-activated channel activity. We therefore assessed effects of RNAi silencing of IP3 receptor, NMDA receptor, and SERCA1 on isoflurane-induced caspase-3 activation in H4-APP cells. Further, described herein are the effects of memantine administration on isoflurane-induced caspase-3 activation both in H4-APP cells and in naive mice.

### Postoperative Cognitive Dysfunction

AD is associated with cognitive impairment and it has been suggested that some mechanisms underlying AD manifestation are also underlying some forms of cognitive impairment, for example apoptosis, altered processing of APP, elevated Aβ levels, etc.

A cognitive impairment experienced after a clinical intervention is termed postoperative cognitive dysfunction (POCD). POCD is commonly observed after inhalation anesthesia, for example after anesthesia with a halogenated ether, such as isoflurane. POCD refers to cognitive problems, for example with memory, learning, or the ability to concentrate following a clinical intervention. The absorption, processing, retainment and/or output of information by the subject may be impaired in POCD. POCD can manifest as a short-term symptom, or last for extended periods of time. Some patients were reported to demonstrate POCD at periods longer than 1 year after a clinical intervention, suggesting that in certain subjects at risk of developing POCD, POCD may be or cause a permanent alteration of cognitive function.

POCD has been studied through various institutions, for example in the IPOCDS-I study centered in Eindhoven, Netherlands and Copenhagen, Denmark. This study through logical longitudinal extrapolation found that age, duration of anesthesia, and introperative complications to be risk factors for POCD. Examples of other risk factors for POCD have been identified, such as history of head injury, cardiovascular disease, poor or impaired cognition, and others mentioned herein.

POCD risk factors can be assessed in subjects in need of a clinical intervention, for example anesthesia, and subjects can be identified to be at risk of developing POCD by the results of the assessment of such risk factors. Methods for assessing POCD risk factors, for example assessment of age, history of head injury, cardiovascular health, cognition, and general health are well known to those of skill in the related medical arts.

### Uncompetitive NMDA Receptor Antagonists

Uncompetitive NMDA receptor antagonists are a class of anesthetics that antagonize, or inhibit the activity of, the N-methyl d-aspartate (NMDA) receptor. They are used in anesthesia for animals and, less commonly, for humans. The state of anesthesia they induce is referred to as dissociative anesthesia. Examples for uncompetitive NMDA receptor antagonists are Memantine, Ketamine, Amantadine, Dextromethorphan, Dextrorphan, Ibogaine, Nitrous oxide, Phencyclidine, Riluzole, and Tiletamine. Described herein are methods for the use of uncompetitive NMDA receptor antagonists to improve inhalational anesthesia, for example by ameliorating or preventing certain undesired side effects, including, for example an increased risk to develop AD or a cognitive impairment.

### Memantine

Memantine is a relatively new FDA-approved drug for the treatment of AD, which acts as an uncompetitive (partial) antagonist of the NMDA receptor (for review, see Lipton, 2006). Memantine is the first in a novel class of AD medications acting on the glutamatergic system by blocking NMDA glutamate receptors. Memantine was first synthesized and patented by Eli Lilly and Company in 1968 (see, e.g., Merck Index). Memantine is marketed, for example, under the brands Axura and Akatinol by Merz, Namenda by Forest , Ebixa and Abixa by Lundbeck and Memox by Unipharm. Memantine is chronically administered to subjects with AD manifestations, for example to treat or ameliorate symptoms of AD. However, memantine has not been suggested for preventative administration either to subjects at risk of developing AD or cognitive impairment, or being administered in temporal proximity to a clinical intervention known to be associated with elevated risk of developing AD or cognitive impairment. Some embodiments of this invention provide methods of administering memantine in temporal proximity to, for example before and/or during, the administration of such clinical interventions, for example administration of inhalation anesthetics. As used herein, “in temporal proximity to” means that the treatments (e.g., administration of memantine and an inhalation anesthetic) are administered, in either order, within 6 hours of each other, within 5 hours of each other, within 4 hours of each other, within 3 hours of each other, within 2 hours of each other, within 1 hour of each other, within 30 minutes of each other, within 20 minutes of each other, within 10 minutes of each other, or substantially simultaneously.

In some embodiments, the uncompetitive NMDA receptor antagonist, for example memantine, is administrated in an amount sufficient to decrease a level of amyloid-β or to prevent an increase in a level of amyloid-β in a cell of said subject. In some embodiments, the uncompetitive NMDA receptor antagonist is administrated in an amount sufficient to prevent apoptosis in a cell in said subject. In some embodiments, the uncompetitive NMDA receptor antagonist is administrated in an amount sufficient to prevent or inhibit a cognitive impairment or to decrease the level of a cognitive impairment in said subject. The term “sufficient amount” is an amount sufficient to provide an observable improvement over the baseline clinically observable state without the administration of the uncompetitive NMDA receptor antagonist. Methods for the assessment of Aβ levels, the assessment of apoptosis, the assessment of cognition, are well known to those of ordinary skill in the art. Sufficient amounts, or doses, of commercially available formulations of uncompetitive NMDA receptor antagonists, are for example memantine, marketed as Axura and Akatinol by Merz, Namenda by Forest, Ebixa and Abixa by Lundbeck and Memox by Unipharm, have been established during clinical trials and are well known to those of skill in the medical arts, in which such formulations are routinely administered to patients. A sufficient amount and/or the optimal dose of the uncompetitive NMDA receptor antagonist in methods provided herein can be determined empirically for each individual of for classes of individuals using well-known methods and will depend upon a variety of factors, including the activity of the agents; the age, body weight, general health, gender and diet of the individual; the time and route of administration; and other medications the individual is taking. Sufficient amounts and optimal dosages may be established using routine testing and procedures that are well known in the art.

### Inhibitory Nucleic Acids

In certain embodiments inhibitors of gene expression can be used for the methods described herein, such as siRNAs specific for a IP3 receptor gene transcript or a SERCA1 gene transcript, wherein the siRNAs reduce the amount of IP3 receptor or SERCA1 mRNA and IP3 receptor or SERCA1 protein in the subject. Use of such inhibitory nucleic acids is demonstrated in the Examples to reduce the deleterious effects of the inhalation anesthetic isoflurane.

Inhibitory nucleic acid molecules that are short interfering nucleic acids (siNA), which include, short interfering RNA (siRNA), double-stranded RNA (dsRNA), micro-RNA (miRNA), and short hairpin RNA (shRNA) molecules are used to inhibit the expression of target genes, such as IP3 receptor or SERCA1. The siNAs of the present invention, for example siRNAs, typically regulate gene expression via target RNA transcript cleavage/degradation or translational repression of the target messenger RNA (mRNA). In one embodiment siRNAs are exogenously delivered to a cell. In a specific embodiment siRNA molecules are generated that specifically target IP3 receptor or SERCA1.

A short interfering nucleic acid (siNA) of the invention can be unmodified or chemically-modified. A siNA of the instant invention can be chemically synthesized, expressed from a vector or enzymatically synthesized. The instant invention also features various chemically-modified synthetic short interfering nucleic acid (siNA) molecules capable of inhibiting gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. For example, in some cases, siRNAs are modified to alter potency, target affinity, the safety profile and/or the stability to render them resistant or partially resistant to intracellular degradation. Modifications, such as phosphorothioates, for example, can be made to siRNAs to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluyl nucleotides maintain gene silencing activity (Xia et al., ASC Chem. Biol. 1(3):176-83, (2006). siRNAs with amide-linked oligoribonucleosides have been generated that are more resistant to S1 nuclease degradation (Iwase R et al. 2006 Nucleic Acids Symp Ser 50: 175-176). In addition, modification of siRNA at the 2′-sugar position and phosphodiester linkage confers improved serum stability without loss of efficacy (Choung et al., Biochem. Biophys. Res. Commun. 342(3):919-26, 2006). In one study, 2′-deoxy-2′-fluoro-beta-D-arabinonucleic acid (FANA)-containing antisense oligonucleotides compared favorably to phosphorothioate oligonucleotides, 2′-O-methyl-RNA/DNA chimeric oligonucleotides and siRNAs in terms of suppression potency and resistance to degradation (Ferrari N et a. 2006 Ann NY Acad Sci 1082: 91-102).

The present invention contemplates in vitro use of TDO2 siRNAs (shRNAs, etc.) as well as in vivo pharmaceutical preparations containing siRNAs (shRNAs, etc.) that may be modified siRNAs (shRNAs, etc.) to increase their stability and/or cellular uptake under physiological conditions, that specifically target nucleic acids encoding IP3 receptor or SERCA lenzyme, together with pharmaceutically acceptable carriers.

### Formulations

The therapeutic, such as an uncompetitive NMDA receptor antagonist, for example memantine, or an inhibitory nucleic acid, will be formulated as a pharmaceutically acceptable salt in some embodiments of this invention. The term “pharmaceutically acceptable salt” includes derivatives of the disclosed uncompetitive NMDA receptor antagonists, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, and nitric; other acceptable inorganic salts include metal salts such as sodium salt, potassium salt, and cesium salt; and alkaline earth metal salts, such as calcium salt and magnesium salt; and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4; organic amine salts such as triethylamine salt, pyridine salt, picoline salt, ethanolamine salt, triethanolamine salt, dicyclohexylamine salt, N,N′-dibenzylethylenediamine salt; and amino acid salts such as arginate, asparginate, and glutamate, and combinations comprising one or more of the foregoing salts.

### Pharmaceutical Formulations, Kits and Routes of Administration

Provided herein are pharmaceutical formulations and kits comprising a combination of agents for improved inhalation anesthesia with a halogenated ether as described herein. Anesthetics for use in such formulations and kits include isoflurane, enflurane, halothane, sevoflurane or desflurane. Also included in the formulations and kits are one or more uncompetitive NMDA receptor antagonists, such as memantine, and/or one or more chelating agents such as: acrylic polymers, ascorbic acid, BAPTA (1,2-bis (o-aminophenoxy)ethane-N,N,N′,N′-tetraacetic acid), BayPure® CX 100 (tetrasodium iminodisuccinate), citric acid, dicarboxymethylglutamic acid, ethylenediaminedisuccinic acid (EDDS), ethylenediaminetetraacetic acid (EDTA), hepta sodium salt of diethylene triamine penta (methylene phosphonic acid) (DTPMP.Na7), malic acid, nitrilotriacetic acid (NTA), nonpolar amino acids, such as methionine, oxalic acid, phosphoric acid, polar amino acids, including: arginine, asparagine, aspartic acid, glutamic acid, glutamine, lysine, and ornithine, siderophores such as Desferrioxamine B, and succinic acid.

The pharmaceutical formulations and kits may additionally comprise a carrier or excipient, stabilizer, flavoring agent, and/or coloring agent. Kits may include instructions for administering the anesthetic and uncompetitive NMDA receptor antagonist (s) in a particular sequence or with a specific or recommended timing.

The uncompetitive NMDA receptor antagonists provided herein may be administered using a variety of routes of administration, for example enteral or parenteral routes, known to those skilled in the art. Routes of administration include oral administration, for example in immediate release or extended release formulations. In some embodiments, a pharmaceutical formulation comprising an uncompetitive NMDA receptor antagonist may be taken orally in the form of liquid, syrup, tablet, capsule, powder, sprinkle, chewtab, or dissolvable disc. Alternatively, pharmaceutical formulations of the present invention can be administered intravenously or transdermally. Additional routes of administration known to those skilled in the art (see, e.g., Remington's Pharmaceutical Sciences, Gennaro A. R., Ed., 20^{th} Edition, Mack Publishing Co., Easton, Pa.).

### Identification of Subjects at Increased Risk of Developing AD or Cognitive Dysfunction

Improved methods of anesthesia provided herein are especially useful in the treatment of subjects at risk to develop AD or a cognitive impairment. Risk factors for developing AD and cognitive impairment are well established in the art.

Well known risk factors that increase the likelihood of developing AD and/or a cognitive dysfunction are, for example, age, family history, genetic factors, brain health and general health.

One of the best established factors determining risk to develop AD or cognitive dysfunction is age. Most individuals with AD manifestation are 65 years old and older and the risk approximately doubles every five years after the age of 65. Therefore, a subject of 65 years and older can be identified as a subject at risk of developing AD. A similar age-risk relation exists for the development of a cognitive dysfunction.

Likewise, those subjects having a parent, brother or sister, or child with AD or a cognitive dysfunction are at increased risk develop AD or a cognitive dysfunction themselves. The risk increases further if more than one family member has the disease or condition. When diseases or conditions tend to run in families, either heredity or environmental factors or both may play a role.

Genes involved in AD risk are well known in the art. Such AD “risk genes” increase the risk of developing AD, but do not determine that the disease will manifest. One example of a well-established AD risk gene is apolipoprotein E-e4 (APOE-e4). APOE-e4 is one of three common forms, or alleles, of the APOE gene; the others are APOE-e2 and APOE-e3. APOE provides the blueprint for one of the proteins that carries cholesterol in the bloodstream. Everyone inherits a copy of some form of APOE from each parent. Those who inherit one copy of APOE-e4 have an increased risk of developing AD. Those who inherit two copies have an even higher risk, but not a certainty of developing AD. In addition to raising risk, APOE-e4 may tend to make symptoms appear at a younger age than usual. Other AD risk genes in addition to APOE-e4 are well established in the art.

Genetic tests are well established in the art and are available, for example for APOE-e4. A subject carrying the APOE-e4 allele can, therefore, be identified as a subject at risk of developing AD.

Other risk factors for AD and cognitive dysfunction have been established, for example head injury, the quality of heart-head connection, cardiovascular health, and general health.

There appears to be a strong link between serious head injury and future risk of AD and/or cognitive dysfunction. A subject with a history of head injury can, therefore, be identified as a subject at risk of developing AD or cognitive dysfunction.

Some of the strongest evidence links brain health to heart health. The brain is nourished by one of the body's richest networks of blood vessels. The risk of developing AD or cognitive dysfunction appears to be increased by many conditions that damage the heart or blood vessels. These include high blood pressure, heart disease, stroke, diabetes and high cholesterol. A subject suffering from any of these conditions can, therefore be identified as a subject at increased risk of developing AD or cognitive dysfunction.

Other lines of evidence suggest that general health condition is a determinant for the risk of developing AD or a cognitive dysfunction. A subject in bad general health, for example an overweight subject, a heavy drinker or smoker, etc., may be identified as a subject at risk of developing AD and/or a cognitive dysfunction.

### Examples

### Materials and Methods

Cell lines. We used naive H4 human neuroglioma cells stably transfected to express full-length APP (FL-APP; H4-APP cells) in the studies. The cells were cultured in DMEM (high glucose) containing 9% heat inactivated fetal calf serum, 100 U/ml penicillin, 100 μg/ml streptomycin, 2 mM L-glutamine, and 200 μg/ml G418.

Cell treatment. Twenty-one percent O2, 5% CO2, and 2% isoflurane were delivered from an anesthesia machine to a sealed plastic box in a 37° Celsius incubator containing six-well plates seeded with one million cells in 1.5 ml of cell culture media as described in our previous studies (Xie et al., 2006a,b, 2007). Date infrared gas analyzer (Puritan-Bennett, Tewksbury, Mass.) was used to continuously monitor the delivered CO2, O2, and isoflurane concentrations. We treated the cells with 2% isoflurane for 6 h, during which time the cells were incubated in serum-free media. In the interaction studies, the cells were treated with memantine (4 μM) 1 h before the treatment of 2% isoflurane. Low-calcium condition was created by using a calcium-free cell culture media (Invitrogen, Carlsbad, Calif.) and by adding EDTA (10 mM; Sigma-Aldrich, St. Louis, Mo.), a chelating compound (Rekasi et al., 2005), or BAPTA (10 μM; Sigma-Aldrich), an intracellular calcium chelator, to the media. To avoid off target effects of RNAi, we used two sets of small interference RNAs (siRNAs) aimed at knockdown of NMDA receptor (NR)1 (first set, 3′GCCGGGAUCUUCCUGAUUUUU, 5′-PAAAUCAGGAAGAUCCCGGCUU; second set, 3′GGAGCACGCUGGACUCGUUUU, 5′PAACGAGUCCAGCGUGCUCCUU), IP3 receptor (first set, 3′GCAAUCACAUGUGGAAAUUUU, 5′-AAUUUCCACAUGUGAUUGCUU; second set, 3′UGGAAAGUCUGACCGAAUAUU, 5′PUAUUCGGUCAGACUUUCCAUU), and SERCA (first set, 3′ UCGCACAAGUCCAAGAUUGUU, 5′-CAAUCUUGGACUUGUGCGAUU; second set, 3′GGCCAAAGGUGUCUAUGAGUU, 5′-PCUCAUAGACACCUUUGGCCUU). These siRNAs and control siRNAs (3′UAGCGACUAAACACAUCAAUU) were obtained from Dharmacon (Lafayette, Colo.). siRNAs were transfected into cells by using electroporation (Amaxa Biosystems, Gaithersburg, Md.) as described by Xie et al. (2005b). Briefly, we mixed 1 million cells, 100 μl of AMAXA electroporation transfection solution, and 10 μl of 20 μM siRNA together and then used the C-9 program in an AMAXA electroporation device for cell transfection. The transfected cells were then placed in one of the six-well plates containing 1.5 ml of cell culture media. The pretreated cells were then exposed to the isoflurane treatment.

Cell lysis and protein amount quantification. Cell pellets were detergent-extracted on ice using immunoprecipitation buffer (10 mM Tris-HCl, pH 7.4, 150 mM NaCl, 2 mM EDTA, 0.5% Nonidet P-40) plus protease inhibitors (1 μg/ml aprotinin, 1 μg/ml leupeptin, 1 μg/ml pepstatin A). The lysates were collected, centrifuged at 12,000 rpm for 10 min, and quantified for total proteins by a BCA protein assay kit (Pierce Technology Corporation, Iselin, N.J.).

Cytosolic calcium measurement. Cytosolic calcium levels were determined as described by Wei et al. (2008). Specifically, H4-APP cells were loaded with Fura-2 (Invitrogen), perfused with Tyrode buffer, and [Ca2+]i transients were recorded as changes in Fura-2 ratio (340/380 nm) using a spectrofluoroscope system (Ionoptix, Milton, Mass.). The cells were exposed to isoflurane (1 mM or 3.5%) with or without pretreatment with memantine (4 μM).

Mice treatment. The animal protocol was approved by the Massachusetts General Hospital Standing Committee on Animals Sixteen female C57BL/6 mice (5-6 months of age) (The Jackson Laboratory, Bar Harbor, Me.) were randomly assigned to an anesthesia or control group. Mice randomized to the anesthesia group (n=8) received three sessions of 1.4% isoflurane in 100% oxygen for 2 h in an anesthetizing chamber. The mice were placed in 100% oxygen without isoflurane in the chamber for 10 min between each session. This isoflurane treatment allows the mice to have sufficient exposure to isoflurane while avoiding excessive accumulation of CO2. The control mice (n=8) received 100% oxygen at an identical flow rate for 6 h and 20 min in an identical chamber. The mice breathed spontaneously, and anesthetic and oxygen concentrations were measured continuously (Datex, Puritan-Bennett; Ohmeda PPD, Madison, Wis.). Rectal temperature was measured intermittently, and the temperature of the anesthetizing chamber was controlled to maintain rectal temperature of the animals at 37±0.5° C. Mean arterial blood pressure was measured noninvasive using a tail cuff (CODA2 system; Kent Scientific Corporation, Torrington, Conn.) in the anesthetized mice. This treatment of isoflurane did not significantly affect the blood pressure or blood gas of the mice (data not shown). Mice were killed by decapitation at the end of the experiments, the brain was removed rapidly, and the prefrontal cortex was dissected out and frozen in liquid nitrogen for subsequent processing for the determination of caspase-3activation. For interaction studies, half of the anesthetized mice and half of the control mice received memantine (20 mg/kg) by intraperitoneal injection 1 h before the isoflurane treatment (Chen et al., 1998).

Brain tissue lysis and protein amount quantification. The harvested brain tissues were homogenized on ice using immunoprecipitation buffer (10 mM Tris-HCl, pH 7.4, 150 mM NaCl, 2 mM EDTA, 0.5% Nonidet P-40) plus protease inhibitors (1 μg/ml aprotinin, 1 μg/ml leupeptin, 1 μg/ml pepstatin A). The lysates were collected, centrifuged at 12,000 rpm for 10 min, and quantified for total proteins by a BCA protein assay kit (Pierce Technology Corporation).

Western blot analysis. The harvested cells and brain tissues were subjected to Western blots as described by Xie et al. (2005a). A caspase-3 antibody (1:1000 dilution; Cell Signaling Technology, Beverly, Mass.) was used to recognize caspase-3 fragment (17-20 kDa) resulting from cleavage at asparate position 175 and FL-caspase-3 (35-40 kDa). Caspase-3 cleavage (activation) is based on the ratio of caspase-3 fragment to FL caspase-3. Antibody anti-b-actin was used to visualize b-actin (42 kDa). Antibodies for IP3 receptor (1:1000; Millipore Bioscience Research Reagents, Temecula, Calif.), NR1 (1:1000; PhosphoSolutions, Aurora, Colo.), and SERCA1 (1:1000; Abcam, Cambridge, Mass.) were used to recognize IP3 receptor (250 kDa), NR1 (120 kDa), and SERCA1 (110 kDa). The quantitation of Western blots was performed as described by Xie et al. (2005a). Briefly, the intensity of signals was analyzed by using a Bio-Rad (Hercules, Calif.) image program (Quantity One) and an NIH Image version 1.37v. We quantified the Western blots using two steps. First, we used levels of b-actin to normalize (e.g., determining the ratio of FL-caspase-3 amount to b-actin amount) levels of proteins to control for loading differences in total protein amounts. Second, we presented changes in levels of proteins in the mice or cells treated with isoflurane as the percentage of those in the mice or cells treated with controls.

Cell viability assay. 3- (4,5-Dimethylthiazol-2-yl)2,5diphenyltetrazoliumbromide (MTT, Thiazolyl blue) is a water soluble tetrazolium salt. Dissolved MTT is converted to the insoluble purple formazan by cleavage of the tetrazolium ring by dehydrogenase enzymes. Active mitochondrion dehydrogenase (only in viable cells) will cause this conversion. We used a MTT assay kit (Sigma-Aldrich) to measure cell viability. The absorbency was measured with a spectrophotometer at a wavelength of 570 nm with background subtraction at 630 nm. Cell viability reduction suggests cell death.

Cell apoptosis assay. Cell apoptosis was assessed by a cell death detection ELISA kit (Roche, Palo Alto, Calif.), which assays cytoplasmic histone associated DNA fragmentation associated with cellular apoptosis.

Statistics. Changes in caspase-3 activation were presented as a percentage of those of the control group. One hundred percent caspase-3 activation refers to control levels for purposes of comparison with experimental conditions. Data were expressed as mean±SD. The number of samples varied from 3 to 10, and the samples were normally distributed. We used two-tailed t test to compare the difference between the experimental groups and control groups. p values ≦0.05 (* or #) and 0.01 (** or ##) were considered statistically significant.

### Example 1

### Memantine Attenuates Isoflurane Induced Caspase-3 Activation, Cell Viability Reduction, and Apoptosis in H4-APP Cells

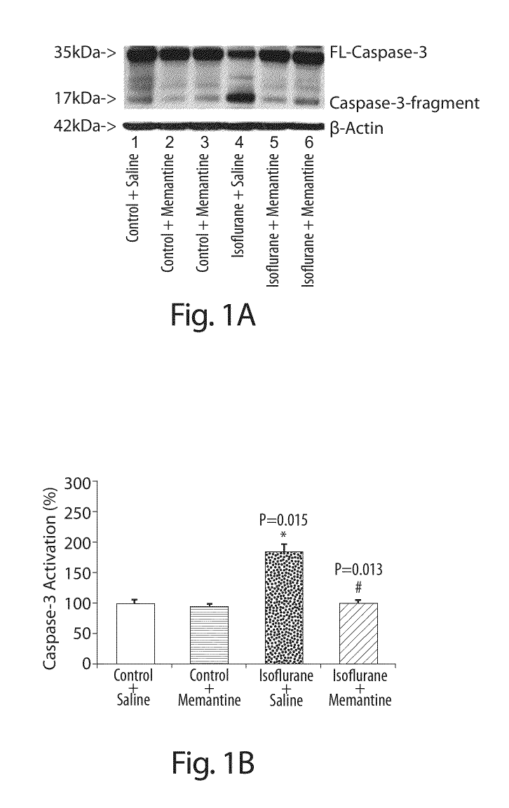

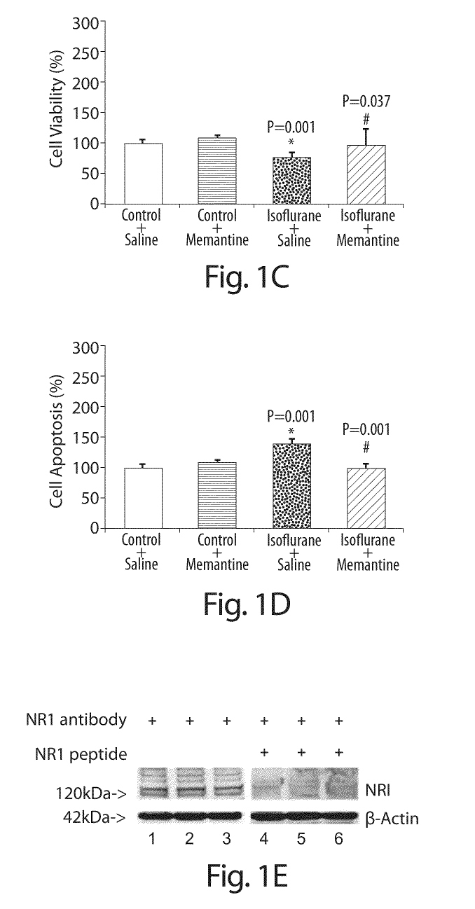

We reported previously that isoflurane can induce caspase-3 cleavage (activation) in H4-APP cells (Xie et al., 2006a,b, 2007). The underlying molecular mechanisms, however, remain primarily to be determined Recent studies have shown that elevated levels of cytosolic calcium released from the ER may be at least partially responsible for isoflurane-induced caspase-3activation and apoptosis (Wei et al., 2005, 2008). Memantine, a newly FDA approved drug for AD (Lipton, 2006), is a partial antagonist for the NMDA receptor, which can reduce influx of extracellular calcium. We therefore set out to assess whether memantine attenuate isoflurane-induced caspase activation in H4-APP cells. Because caspase activation alone cannot report cell death (McLaughlin et al., 2003), we also monitored effects of isoflurane and memantine on cell viability and apoptosis in H4-APP cells Immunoblotting for caspase-3 revealed increases in caspase-3 fragment and decreases in FL-caspase-3 in H4-APP cells treated with 2% isoflurane compared with those cells in control conditions (FIG. 1A). Treatment with memantine plus isoflurane led to a visible decrease in caspase-3 activation compared with isoflurane treatment alone (FIG. 1A). Memantine treatment alone did not induce caspase-3 activation compared with control conditions (FIG. 1A). Quantification of the results showed that isoflurane treatment led to a 184% increase in caspase-3 activation compared with control conditions (FIG. 1B) (p=0.015). Memantine plus isoflurane yielded a significant reduction in isoflurane-induced caspase-3 activation (FIG. 1B): 101 versus 184% (p=0.013).These results indicate that a clinically relevant concentration (2%) of isoflurane can induce apoptosis in H4-APP cells, and a clinically relevant concentration of memantine can attenuate isoflurane-induced caspase-3 activation (apoptosis). Memantine alone did not increase caspase-3 activation compared with control conditions (FIG. 1B). MTT cell viability and cell apoptosis studies showed that isoflurane treatment led to a 26% reduction in cell viability (FIG. 1C) (p=0.001) and a 137% increase in cell apoptosis (FIG. 1D) (p=0.001) in H4-APP cells, compared with control conditions, respectively. Memantine treatment significantly attenuated the isoflurane-induced reduction in cell viability (FIG. 1C), 74 versus 95% (p=0.037), and isoflurane-induced apoptosis (FIG. 1D), 137 versus 99% (p=0.001). Meanwhile, memantine treatment alone had no effects on either cell viability or apoptosis compared with control conditions in H4-APP cells. These results suggest that isoflurane can induce caspase-3 activation, cell viability reduction, and apoptosis in H4-APP cells, and memantine can attenuate isoflurane-induced apoptotic cell damage, conceivably via antagonism of NMDA receptors. Furthermore, immunoblotting for NR1 specifically revealed that there are NMDA receptors in H4-APP cells (FIG. 1E), the cell lines in which memantine was able to attenuate effects of isoflurane on caspase-3 activation, cell viability reduction, and apoptosis. These findings further suggest that memantine may act on NMDA receptors to inhibit isoflurane-induced apoptotic cell damage.

### Example 2

### Memantine Inhibits Isoflurane-Induced Elevation in Cytosolic Calcium Levels in H4-APP Cells

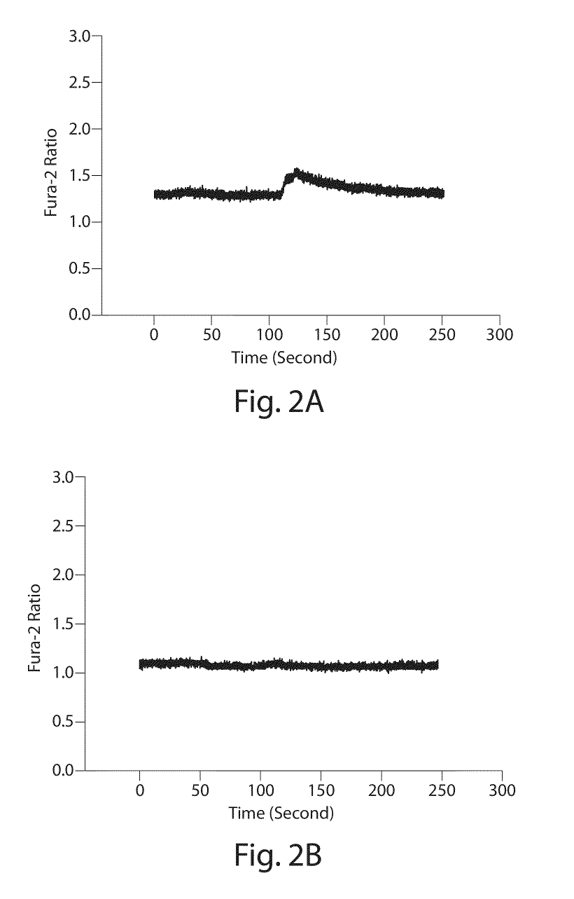

Given that excessive cytosolic calcium levels can trigger caspase activation and apoptosis (for review, see Mattson, 2007), and isoflurane may induce apoptosis via elevation of cytosolic calcium levels (Wei et al., 2005, 2008), we next asked whether memantine can inhibit the isoflurane-induced elevation in cytosolic calcium levels in H4-APP cells. We found that the isoflurane (1 mM or 3.5%) treatment can elevate cytosolic calcium levels in H4-APP cells (FIG. 2A). In contrast, in the H4-APP cells pretreated with memantine (4 μM), isoflurane failed to increase cytosolic calcium levels (FIG. 2B). These results suggest that memantine may inhibit the isoflurane-induced elevation in cytosolic calcium to attenuate isoflurane-induced caspase activation and apoptosis.

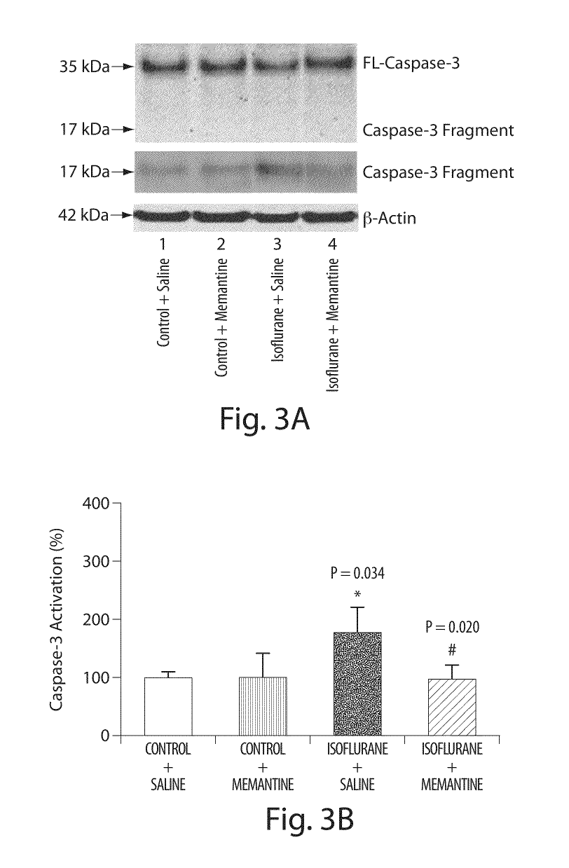

Memantine attenuates isoflurane-induced caspase-3activation in mouse brain. We next set out to assess the effects of memantine on isoflurane induced caspase-3 activation in vivo in naive mice. After establishing that treatment with 1.4% isoflurane for 2 h did not significantly affect blood pressure and venous blood gas (data not shown), we exposed the mice to 1.4% isoflurane treatment for three 2 h sessions with 10 min between each session. This treatment enabled the mice to have enough isoflurane exposure yet avoid severe CO2 accumulation, because the mice reach a fully awake status between each session. The three sessions of 1.4% isoflurane for 2 h inducedcaspase-3 activation in the mouse brain compared with the control conditions (FIG. 3A). Treatment with memantine (20 mg/kg, intraperitoneal route) plus isoflurane (three sessions of the treatment with 1.4% isoflurane for 2 h) led to a visible decrease in caspase-3 activation compared with isoflurane treatment alone (FIG. 3A). Treatment with memantine (20 mg/kg, intraperitoneal route) alone did not induce caspase-3 activation compared with the control condition (FIG. 3A). Quantification of these results showed that the isoflurane treatment led to 177% increase in caspase-3 activation compared with control condition (FIG. 3B) (p=0.034). Treatment with memantine plus isoflurane significantly reduced caspase-3 activation compared with isoflurane treatment alone (FIG. 3B) (p=0.020; 177 vs. 97%). Memantine treatment alone did not inducecaspase-3 activation compared with the control condition (FIG. 3B). Collectively, all of these findings showed that the NMDA receptor partial antagonist, memantine, can attenuate isoflurane induced apoptosis both in vivo and in vitro.

### Example 3

### RNAi Knockdown of NR1 Reduces Isoflurane-Induced Caspase-3 Activation

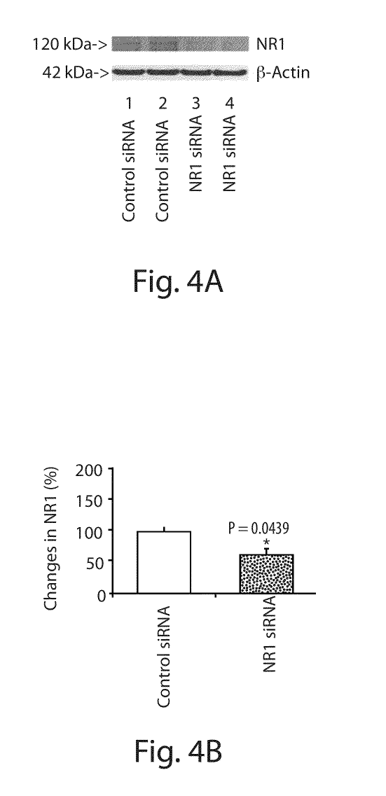

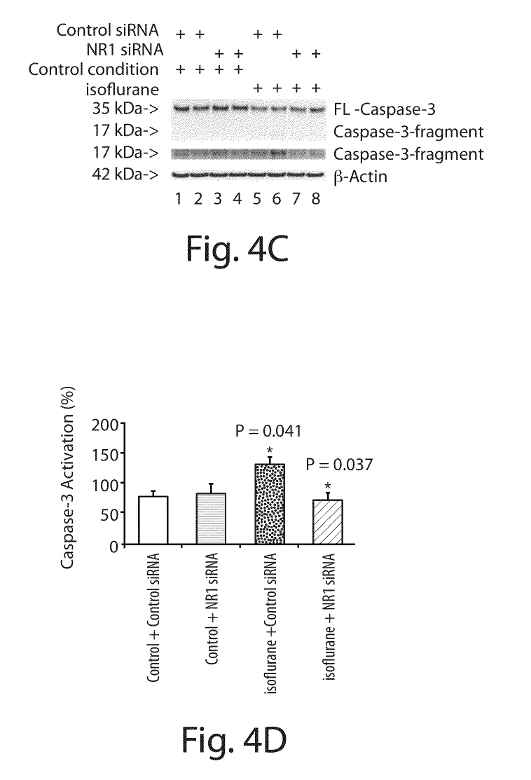

Given that memantine can attenuate isoflurane-induced apoptosis, we next asked whether reductions in protein levels of NMDA receptors could also decrease isoflurane-induced apoptosis. We therefore assessed the effects of RNAi-mediated knockdown of NR1 on isoflurane-induced caspase-3 activation in H4-APP cells. RNAi silencing of NR1 significantly reduced NR1 levels (FIGS. 4A,B), 100 versus 62% (p=0.0439). Treatment with 2% isoflurane for 6 h induced caspase-3 activation (FIG. 4C, lanes 5 and 6,D, black) (p=0.041). RNAi knockdown of NR1 (FIG. 4C, lanes land 8, D, lined) attenuated isoflurane-induced caspase-3 activation in H4-APP cells, 168 versus 93% (p=0.037). We repeated these experiments with different NR1 siRNAs and observed similar effects: RNAi knockdown of NR1 attenuated isoflurane induced caspase-3 activation in H4-APP cells (data not shown). These findings suggest that the effects of RNAi knockdown of NR1 on isoflurane-induced caspase-3 activation are not likely to be caused by off-target effects of RNAi silencing of NR1. Collectively, these findings suggest that NR1 plays a role in isoflurane induced caspase activation and apoptosis.

### Example 4

### Reduced Extracellular and Intracellular Calcium Levels Attenuate Isoflurane-Induced Caspase-3 Activation

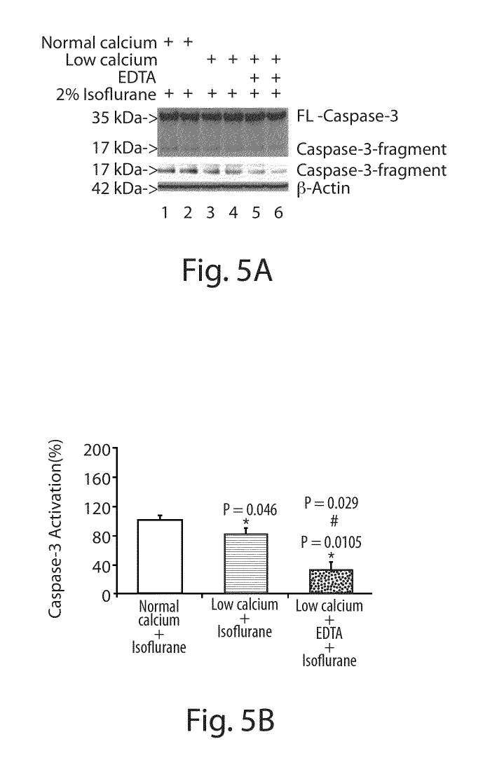

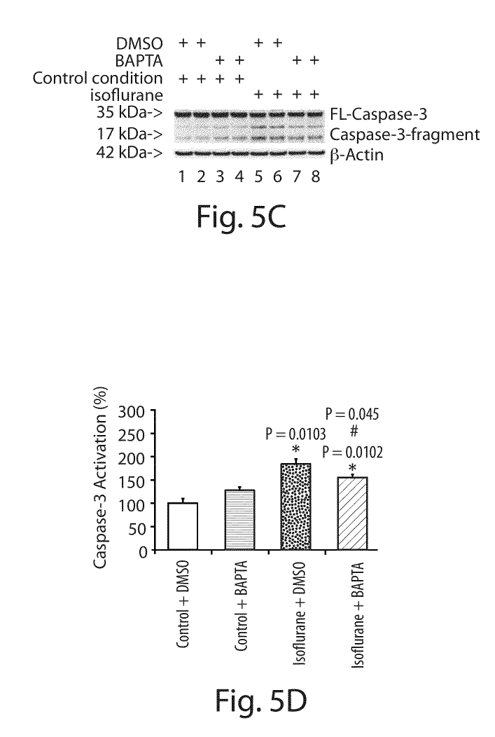

Excitotoxic neuronal cell damage can be exacerbated by hyperactivation of NMDA receptors, which results in excessive calcium influx and subsequent free radical formation (for review, see Lipton, 2006). Isoflurane has been reported to induce apoptosis by elevating cytosolic calcium levels (Wei et al., 2005). Given that memantine and RNAi knockdown of NR1 attenuated isoflurane induced apoptosis, we next addressed whether this involves partial inhibition of calcium influx by exposing H4-APP cells to low-calcium levels and/or treating with the chelator EDTA. Treatment with isoflurane plus low calcium led to a visible reduction in caspase-3 activation compared with treatment with isoflurane under normal calcium conditions (FIG. 5A). Treatment with isoflurane plus low calcium plus EDTA led to even less caspase-3activation compared with either isoflurane alone or isoflurane plus low calcium (FIG. 5A). Quantitation of these results revealed that treatment with isoflurane plus low calcium (FIG. 5B, gray) led to a 19% reduction (p=0.046) in caspase-3 activation compared with isoflurane plus normal calcium condition (FIG. 5B, white).Treatment with isoflurane plus low calcium and EDTA (FIG. 5B, black) led to an even greater (68%; p=0.015) reduction in caspase-3 activation compared with the treatment with isoflurane plus normal calcium (FIG. 5B, white). To further confirm that isoflurane-induced caspase-3 activation is associated with cytosolic calcium levels, we assessed effects of BAPTA, an intracellular calcium chelator, on isoflurane induced caspase-3 activation in H4-APP cells. Treatment with 2% isoflurane for 6 h induced caspase-3 activation (FIG. 5C, lanes 5 and 6,D, black) (p=0.0102). BAPTA (FIG. 5C, lanes 7 and 8, D, lined) attenuated isoflurane-induced caspase-3 activation in H4-APP cells, 185 versus 154% (p=0.045). Collectively, these results suggest that the isoflurane-induced apoptosis is dependent on both external and cytosolic calcium concentration.

### Example 5

### RNAi Silencing of IP3 Receptor and SERCA1 Attenuate Isoflurane-Induced Caspase-3 Activation

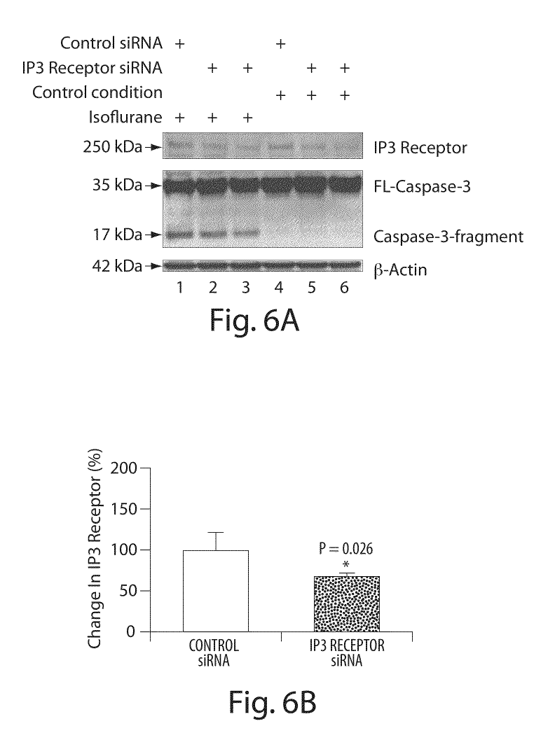

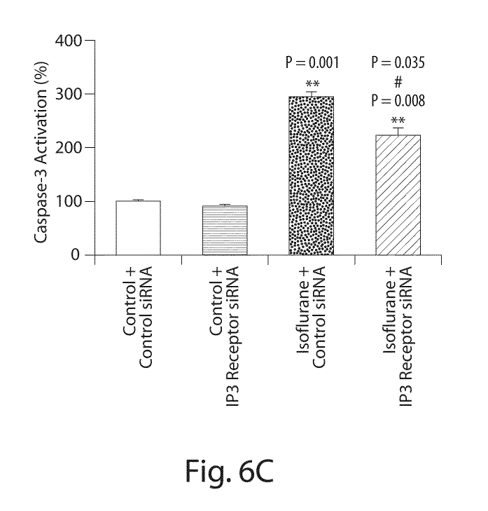

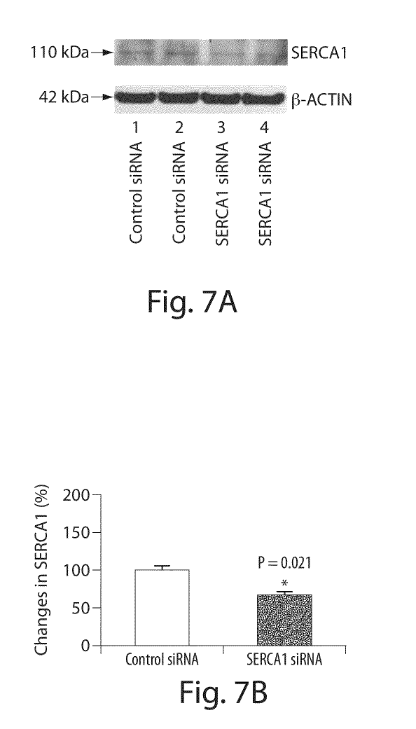

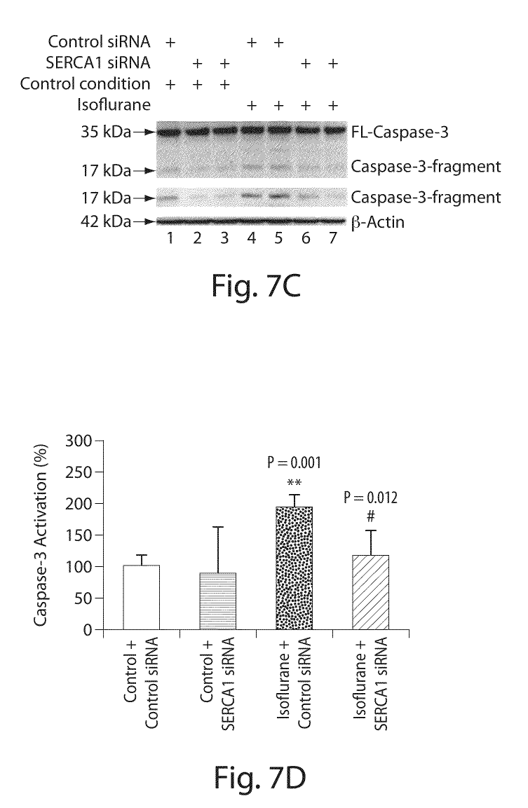

We next asked whether isoflurane-induced apoptosis is dependent on internal calcium released from ER via the IP3 receptor and SERCA1. We first established RNAi knockdown of IP3 receptor in H4-APP cells using siRNA treatment (FIG. 6A). Treatment with isoflurane plus control siRNA led to a visible increase in caspase-3 activation compared with the treatment with control condition plus control siRNA (FIG. 6A). RNAi silencing of IP3 receptor alone did not induce caspase-3 activation compared with the treatment with control condition plus control siRNA (FIG. 6A). However, treatment with isoflurane plus IP3 receptor siRNA attenuated caspase-3 activation compared with the treatment with isoflurane plus control siRNA (FIG. 6A). Quantification of these data showed that RNAi knockdown of IP3 receptor led to a 33% reduction in protein levels of IP3 receptor compared with control siRNA treatment (FIG. 6B) (p=0.026). Treatment with IP3 receptor siRNA alone had no effect on caspase-3 activation (FIG. 6C). Although treatment with isoflurane plus control siRNA led to a 292% increase (p=0.001) in caspase-3 activation compared with the control, treatment with isoflurane plus IP3receptor siRNA (FIG. 6C) led to less caspase-3 activation than did treatment with isoflurane plus control siRNA, 222 versus 292% (p=0.035). These results suggest that knockdown of IP3 receptor via RNAi can attenuate isoflurane-induced caspase-3 activation (apoptosis). Next, we assessed the effects of RNAi-mediated knockdown of SERCA1 on isoflurane-induced caspase-3 activation in H4-APP cells. RNAi-mediated knockdown of SERCA1 reduced SERCA1 levels by 33% (p=0.021) (FIGS. 7A,B). RNAi knockdown of SERCA1 (FIGS. 7C,D) inhibited isoflurane-induced caspase-3 activation in H4-APP cells, 194% (control siRNA) versus 117% (SERCA1 siRNA) (p=0.012). To avoid off-target effects of RNAi knockdown of IP3 receptor and SERCA, we repeated these experiment with different IP3 receptor or SERCA siRNAs and observed similar effects: RNAi knockdown of IP3 receptor and SERCA attenuated isoflurane-induced caspase-3 activation in H4-APP cells (data not shown). Collectively, these findings suggest that isoflurane induction of caspase activation and apoptosis is dependent on calcium release from the ER.

### Discussion

The commonly used inhalation anesthetic isoflurane has been shown previously to induce caspase activation, apoptosis, and enhanced Aβ generation in cultured cells and brain slices (Matsuoka et al., 2001; Jevtovic-Todorovic et al., 2003; Eckenhoffet al., 2004; Loop et al., 2005; Wei et al.,2005; Wise-Faberowski et al., 2005; Xie et al., 2006a,b, 2007). However, the mechanism by which isoflurane induces caspase activation and apoptosis is not clear. It has been suggested that isoflurane may trigger abnormal calcium release from ER to induce caspase activation and apoptosis (Wei et al., 2005, 2008). We show that reductions in extracellular calcium concentration can attenuate isoflurane-induced caspase-3 activation in H4-APP cells, as does RNAi silencing of the IP3 receptor, NR1, and SERCA1. Normal NMDA receptor activity is necessary for induction of long-term potentiation (LTP), a form of synaptic plasticity associated with learning and memory. Hyperactivity of the NMDA receptor, after certain pathological conditions, can engender excessive neuronal calcium influx leading to cellular damage [e.g., apoptosis (for review, see Lipton,2006)]. Here, we have shown that the partial uncompetitive NMDA receptor antagonist memantine can attenuate isoflurane-induced caspase-3 activation in H4-APP cells. Given that caspase-3 activation alone may not suggest cell damage but rather may be essential for neuroprotection in preconditioning (McLaughlin et al., 2003), we also measured effects of isoflurane and memantine on cell viability and apoptosis. We found that memantine can attenuate isoflurane-induced apoptosis and cell viability reduction, which suggests that memantine can inhibit isoflurane-induced apoptotic cell damage. Memantine also attenuated isoflurane-induced elevated cytosolic calcium levels in H4-APP cells and isoflurane-induced caspase-3 activation in the mouse brain in vivo. Collectively, these findings indicate that isoflurane-induced apoptotic cell damage is driven by calcium dyshomeostasis and can be pharmacologically modulated by memantine. Isoflurane has been reported to affect synapse function by acting on the ion channels, including sodium, potassium, and calcium channels, associated with neurotransmitter receptors such as nicotinic, serotonin type 3, GABAA, glycine, and glutamate receptors (Franks and Lieb, 1998; Mennerick et al., 1998; Narahashi et al., 1998) (for review, see Campagna et al., 2003). Specifically, Westphalen and Hemmings (2006) illustrated that isoflurane can enhance basal release of GABA and inhibit basal release of glutamate from isolated rat cortical nerve terminals, and intracellular calcium buffering can limit the isoflurane induced inhibition of basal glutamate release. Hollmann et al. (2001) reported that clinically relevant concentrations of isoflurane, sevoflurane, or desflurane can reduce current associated with NR1/NR2A and NR1/NR2B expressed recombinantly in Xenopus oocytes with a reversible, concentration-dependent, and voltage-insensitive manner. Isoflurane has also been reported to block NMDA-stimulated currents in cultured hippocampus neurons (Yang and Zorumski, 1991) and to attenuate glutamate dependent intraneuronal translocation of Ca2+ (Puil et al.,1990). Recent studies showed that isoflurane can block NMDA receptor-mediated current (Solt et al., 2006), and uncompetitive NMDA receptor antagonist (+)-5-methyl-10,11-dihydro-5H-dibenzo[a,d]cyclohepten-5,10-imine maleate (MK-801) can decrease the minimum alveolar concentration of isoflurane (Eger et al., 2006). In addition, isoflurane has been reported to attenuate LTP in hippocampus slices from mice, and GABA antagonist picrotoxin can inhibit the effects of isoflurane (Simon et al., 2001). Collectively, these findings suggest that isoflurane may inhibit NMDA neurotransmission. However, other studies suggested that isoflurane does not inhibit NMDA transmission (Pearce et al., 1989); in fact, a recent study showed that isoflurane may actually increase NMDA receptor activity by enhancing phosphorylation at S896of the NR1 subunit of NMDA receptor (Zhan et al., 2006). We therefore postulate that isoflurane may affect NMDA neurotransmission with a concentration- and duration-dependent manner. With low concentration and short duration of treatment, isoflurane may inhibit NMDA neurotransmission associated calcium influx to produce neuroprotection effects (e.g., preconditioning). With high concentration and long duration of treatment, however, isoflurane may potentiate NMDA neurotransmission-associated calcium influx to trigger cell damage (e.g., apoptosis). Future studies will be necessary to assess the effects of different concentrations and durations of isoflurane treatments on NMDA neurotransmission and NMDA neurotransmission-associated calcium influx to further test this hypothesis. It is also possible that isoflurane can induce apoptosis via non-NMDA neurotransmission-associated calcium influx. Indeed, El Beheiry et al. (2007) showed that reduction in exogenous calcium concentration and selective blockade of L-type calcium channel with nifedipine can reduce the isoflurane-induced suppression of evoked dendritic field EPSPs in hippocampus slides from old rats. These findings suggest that isoflurane can act on different types of calcium channels to affect cell functions. It will be interesting in future studies to test the effects of various calcium channel blockers, including nifedipine (L-type), ω-conotoxin GVIA (N-type), and ω-conotoxin MVIIC (P/Q-type) (El Beheiry et al., 2007), on isoflurane-induced apoptosis and Aβ generation. Memantine, a newly FDA approved drug for AD treatment, is an uncompetitive (partial) antagonist of the NMDA receptor (for review, see Lipton, 2006). Memantine has a low affinity for the NMDA receptor channel pore and thus has a fast off-rate compared with other uncompetitive NMDA receptor antagonists (e.g., MK-801). This character of memantine enables memantine to only enter the NMDA receptor channel when the channel is opened by antagonist. Therefore, memantine will not accumulate in the NMDA receptor channel and will not interfere with normal synaptic transmission associated with NMDA receptor (Lipton, 1993; Lipton and Rosenberg, 1994; Chen and Lipton, 1997) (for review, see Lipton, 2006). Memantine, an NMDA receptor uncompetitive (partial) antagonist, is different from NMDA noncompetitive antagonists, including MK-801, ketamine, and phencyclidine, in clinical acceptance, because of the fact that memantine can preferentially block NMDA receptor-operated channels when they are excessively open while relatively sparing normal neurotransmission (for review, see Lipton, 2006). Our current findings showing that NMDA receptors exist in H4-APP cells (FIGS. 1E, 4A), and that the NMDA partial antagonist memantine can attenuate isoflurane-induced apoptotic cell damage in H4-APP cells and in naive mice, suggest that memantine may act on NMDA receptors to inhibit isoflurane induced apoptotic cell damage. Moreover, these findings suggest that memantine maybe used to pharmacologically intervene with isoflurane-induced apoptosis and subsequent neurotoxicity in patients, especially elderly and AD patients. Previous studies have suggested that isoflurane can induce apoptosis by activating the ryanodine receptor in the ER to facilitate calcium release from ER to the cytoplasm (Wei et al., 2005). IP3 receptors and SERCA1 can also regulate cytosolic calcium levels (Berridge, 1993; Zhang et al., 2006). Activation of IP3 receptors can cause elevated cytosolic calcium levels, leading to cell death (Hanson et al., 2004; Lindholm et al., 2006). RNAi knockdown of SERCA1 can inhibit calcium influx and calcium release activated calcium channel activity. We therefore assessed the roles of the IP3 receptor and SERCA1 in isoflurane-induced caspase-3 activation. Knockdown of IP3 receptor and SERCA1 in H4-APP cells attenuated isoflurane-induced caspase-3 activation, suggesting that isoflurane can induce apoptosis by activating IP3 receptor and/or SERCA1, facilitating calcium release from the ER to the cytoplasm. Although isoflurane has been reported to induce caspase activation and to cause apoptosis (Matsuoka et al., 2001; Eckenhoff et al., 2004; Kvolik et al., 2005; Loop et al., 2005; Wei et al., 2005; Xie et al., 2006a,b, 2007), other reports suggest that isoflurane can protect against apoptosis (Zaugg et al., 2000; Tyther et al., 2001; Wise-Faberowski et al., 2001; de Klaver et al., 2002; Kawaguchi et al., 2004; Wise-Faberowski et al., 2004; Gray et al., 2005). This difference could be attributable to the use of different cell lines (e.g., rat cardiac cells vs. human neural cells) or differences in duration and concentration of isoflurane exposure (Xie et al., 2006a,b). A transient and moderate elevation of cytosolic calcium levels after a lower concentration of isoflurane treatment for a short duration could provide cytoprotection via up regulating host preconditioning response (Bickler et al., 2005; Bickler and Fahlman, 2006; Zhan et al., 2006). However, prolonged exposure to a high concentration of isoflurane may maintain IP3 receptor in an open status, thereby elevating cytosolic calcium levels and ultimately leading to cell damage (Orrenius et al., 2003; Paschen and Mengesdorf, 2005). Our in vitro and limited in vivo studies as well as other findings suggest that isoflurane may affect AD neuropathogenesis; however, it is necessary to perform further determination of the in vivo relevance of these effects, including in vivo apoptosis (e.g., terminal deoxynucleotidyl transferase-mediated biotinylated UTP nick end labeling) studies, given that caspase activation alone may not represent apoptotic cell damage (McLaughlin et al., 2003), before we can conclude that the inhalation anesthetic isoflurane promotes AD neuropathogenesis in humans. In conclusion, we have shown that decreases in cytosolic calcium levels by reduction in extracellular and intracellular calcium levels, treatment with the NMDA antagonist memantine, or RNAi silencing of the IP3 receptor, NR1, or SERCA1 can attenuate isoflurane-induced apoptosis. These findings suggest a therapeutic strategy for preventing potential isoflurane-associated neurotoxicity (and Aβ generation) based on reducing cytosolic calcium levels. Moreover, we found that this can be achieved pharmacologically by using memantine. In the future studies aimed at further defining the molecular mechanism by which isoflurane induces caspase activation and apoptosis, we will assess effects of isoflurane on cytosolic calcium levels and whether the isoflurane-induced caspase activation and apoptosis are dependent on cytosolic calcium levels. Given the aging of the population and growing numbers of patients with AD who require surgery and general anesthesia, more studies in elucidating the molecular mechanism by which isoflurane induces apoptosis and potentiates Aβ generation are warranted. Our current study indicating that isoflurane-induced apoptosis is dependent on cytosolic calcium levels will hopefully lead to the provision of safer anesthesia care, especially for AD and elderly patients.

### REFERENCES

Berridge M J (1993) Inositol trisphosphate and calcium signaling. Nature 361:315-325.

Bickler P E, Fahlman C S (2006) The inhaled anesthetic, isoflurane, enhances Ca2+-dependent survival signaling in cortical neurons and modulates MAP kinases, apoptosis proteins and transcription factors during hypoxia. Anesth Analg 103:419-429.

Bickler P E, Than X, Fahlman C S (2005) Isoflurane preconditions hippocampal neurons against oxygen-glucose deprivation: role of intracellular Ca2+ and mitogen-activated protein kinase signaling. Anesthesiology 103:532-539.

Bohnen N, Warner M A, Kokmen E, Kurland L T (1994a) Early and midlife exposure to anesthesia and age of onset of Alzheimer's Disease. Int J Neurosci 77:181-185.

Bohnen N I, Warner M A, Kokmen E, Beard C M, Kurland L T (1994b) Alzheimer's disease and cumulative exposure to anesthesia: a case-control study. J Am Geriatr Soc 42:198-201.

Campagna J A, Miller K W, Forman S A (2003) Mechanisms of actions of inhaled anesthetics. N Engl J Med 348:2110-2124.

Chen H S, Lipton S A (1997) Mechanism of memantine block of NMDA activated channels in rat retinal ganglion cells: uncompetitive antagonism. J Physiol (Lond) 499:27-46.

Chen H S, Wang Y F, Rayudu P V, Edgecomb P, Neill J C, Segal M M, Lipton S A, Jensen F E (1998) Neuroprotective concentrations of the N-methyl-D-aspartate open-channel blocker memantine are effective without cytoplasmic vacuolation following post-ischemic administration and do not block maze learning or long-term potentiation. Neuroscience 86:1121-1132.

de Klaver M J, Manning L, Palmer L A, Rich G F (2002) Isoflurane pretreatment inhibits cytokine-induced cell death in cultured rat smooth muscle cells and human endothelial cells. Anesthesiology 97:24-32.

Eckenhoff R G, Johansson J S, Wei H, Carnini A, Kang B, Wei W, Pidikiti R, Keller J M, Eckenhoff M F (2004) Inhaled anesthetic enhancement of amyloid-beta oligomerization and cytotoxicity. Anesthesiology 101:703-709.

Eckert A, Marques C A, Keil U, Schussel K, Muller W E (2003) Increased apoptotic cell death in sporadic and genetic Alzheimer's Disease. Ann NY Acad Sci 1010:604-609.

Eger Jr E I, Liao M, Laster M J, Won A, Popovich J, Raines D E, Solt K, Dutton R C, Cobos Jr F V, Sonner J M (2006) Contrasting roles of the N-methyl-D-aspartate receptor in the production of immobilization by conventional and aromatic anesthetics. Anesth Analg 102:1397-1406.

El Beheiry H, Ouanounou A, Carlen P L (2007) L-type calcium channel blockade modifies anesthetic actions on aged hippocampal neurons. Neuro science 147:117-126.

Franks N P, Lieb W R (1998) Which molecular targets are most relevant to general anaesthesia? Toxicol Lett 100-101:1-8.

Gastard M C, Troncoso J C, Koliatsos V E (2003) Caspase activation in the limbic cortex of subjects with early Alzheimer's Disease. Ann Neuro 154:393-398.

Gervais F G, Xu D, Robertson G S, Vaillancourt J P, Zhu Y, Huang J, LeBlanc A, Smith D, Rigby M, Shearman M S, Clarke E E, Meng H, Van DerPloeg L H, Ruffolo S C, Thornberry N A, Xanthoudakis S, Zamboni R J, Roy S, Nicholson D W (1999) Involvement of caspases in proteolytic cleavage of Alzheimer's amyloid-beta precursor protein and amyloidogenic A beta peptide formation. Cell 97:395-406.

Gray J J, Bickler P E, Fahlman C S, Zhan X, Schuyler J A (2005) Isoflurane neuroprotection in hypoxic hippocampal slice cultures involves increases in intracellular Ca2+ and mitogen activated protein kinases. Anesthesiology 102:606-615.

Hanson C J, Bootman M D, Roderick H L (2004)Cell signalling: IP3 receptors channel calcium into cell death. Curr Biol 14:R933-R935.

Hitomi J, Katayama T, Eguchi Y, Kudo T, Taniguchi M, Koyama Y, Manabe T, Yamagishi S, Bando Y, Imaizumi K, Tsujimoto Y, Tohyama M (2004) Involvement of caspase-4 in endoplasmic reticulum stress-induced apoptosis and Aβ-induced cell death. J Cell Biol 165:347-356.

Hollmann M W, Liu H T, Hoenemann C W, Liu W H, Duryea M E (2001) Modulation of NMDA receptor function by ketamine and magnesium. Part II: interactions with volatile anesthetics. Anesth Analg 92:1182-1191.

Jevtovic-Todorovic V, Hartman R E, Izumi Y, Benshoff N D, Dikranian K, Zorumski C F, Olney J W, Wozniak D F (2003) Early exposure to common anesthetic agents causes widespread neurodegeneration in the developing rat brain and persistent learning deficits. J Neurosci 23:876-882.

Kawaguchi M, Drummond J C, Cole D J, Kelly P J, Spurlock M P, Patel P M (2004) Effect of isoflurane on neuronal apoptosis in rats subjected to focal cerebral ischemia. Anesth Analg 98:798-805.

Kvolik S, Glavas-Obrovac L, Bares V, Karner I (2005) Effects of inhalation anesthetics halothane, sevoflurane, and isoflurane on human cell lines. Life Sci 77:2369-2383.

LeBlanc A, Liu H, Goodyer C, Bergeron C, Hammond J (1999) Caspase-6 role in apoptosis of human neurons, amyloidogenesis, and Alzheimer's disease. J Biol Chem 274:23426-23436.

Lee T A, Wolozin B, Weiss K B, Bednar M M (2005) Assessment of the emergence of Alzheimer's Disease following coronary artery bypass graft surgery or percutaneous transluminal coronary angioplasty. J Alzheimers Dis 7:319-324.

Lindholm D, Wootz H, Korhonen L (2006) ER stress and neurodegenerative diseases. Cell Death Differ 13:385-392.

Lipton S A (1993) Prospects for clinically tolerated NMDA antagonists: open-channel blockers and alternative redox states of nitric oxide. Trends Neurosci 16:527-532.

Lipton S A (2006) Paradigm shift in neuroprotection by NMDA receptor blockade: memantine and beyond. Nat Rev Drug Discov 5:160-170.

Lipton S A, Rosenberg P A (1994) Excitatory amino acids as a final common pathway for neurologic disorders. N Engl J Med 330:613-622.

Loop T, Dovi-Akue D, Frick M, Roesslein M, Egger L, Humar M, Hoetzel A, Schmidt R, Borner C, Pahl H L, Geiger K K, Pannen B H (2005) Volatile anesthetics induce caspase-dependent, mitochondria-mediated apoptosis in human T lymphocytes in vitro. Anesthesiology 102:1147-1157.

Lu D C, Rabizadeh S, Chandra S, Shayya R F, Ellerby L M, Ye X, Salvesen G S, Koo E H, Bredesen D E (2000) A second cytotoxic proteolytic peptide derived from amyloid beta-protein precursor. Nat Med6:397-404.

Matsuoka H, Kurosawa S, Horinouchi T, Kato M, Hashimoto Y (2001) Inhalation anesthetics induce apoptosis in normal peripheral lymphocytes in vitro. Anesthesiology 95:1467-1472.

Mattson M P (2007) Calcium and neurodegeneration. Aging Cell 6:337-350.

McLaughlin B, Hartnett K A, Erhardt J A, Legos J J, White R F, Barone F C, Aizenman E (2003) Caspase 3 activation is essential for neuroprotection in preconditioning. Proc Natl Acad Sci USA 100:715-720.

Mennerick S, Jevtovic-Todorovic V, Todorovic S M, Shen W, Olney J W, Zorumski C F (1998) Effect of nitrous oxide on excitatory and inhibitory synaptic transmission in hippocampal cultures. J Neurosci 18:9716-9726.

Muravchick S, Smith D S (1995) Parkinsonian symptoms during emergence from general anesthesia. Anesthesiology 82:305-307.

Narahashi T, Aistrup G L, Lindstrom J M, Marszalec W, Nagata K, Wang F, Yeh J Z (1998) Ion channel modulation as the basis for general anesthesia. Toxicol Lett 100-101:185-191.

Orrenius S, Zhivotovsky B, Nicotera P (2003) Regulation of cell death: the calcium-apoptosis link Nat Rev Mol Cell Biol 4:552-565.

Paschen W, Mengesdorf T (2005) Endoplasmic reticulum stress response and neurodegeneration. Cell Calcium 38:409-415.

Pearce R A, Stringer J L, Lothman E W (1989) Effect of volatile anesthetics on synaptic transmission in the rat hippocampus. Anesthesiology71:591-598.

Puil E, el-Beheiry H, Baimbridge K G (1990) Anesthetic effects on glutamate-stimulated increase in intraneuronal calcium. J Pharmacol Exp Ther 255:955-961.

Rekasi Z, Czompoly T, Schally A V, Boldizsar F, Varga J L, Zarandi M, Berki T, Horvath R A, Nemeth P (2005) Antagonist of growth hormone releasing hormone induces apoptosis in LNCaP human prostate cancer cells through a Ca2+-dependent pathway. Proc Natl Acad Sci USA 102:3435-3440.

Selkoe D J (2001) Alzheimer's Disease: genes, proteins, and therapy. Physiol Rev 81:741-766.

Simon W, Hapfelmeier G, Kochs E, Zieglgansberger W, Rammes G (2001) Isoflurane blocks synaptic plasticity in the mouse hippocampus. Anesthesiology94:1058-1065.

Solt K, Eger Jr E I, Raines D E (2006) Differential modulation of human N-methyl-D-aspartate receptors by structurally diverse general anesthetics. Anesth Analg 102:1407-1411.

Takuma H, Tomiyama T, Kuida K, Mori H (2004) Amyloid beta peptide induced cerebral neuronal loss is mediated by caspase-3 in vivo. J Neuropathol Exp Neurol 63:255-261.

Tanzi R E, Bertram L (2005) Twenty years of the Alzheimer's Disease amyloid hypothesis: a genetic perspective. Cell 120:545-555.

Tyther R, Fanning N, Halligan M, Wang J, Redmond H P, Shorten G (2001) The effect of the anaesthetic agent isoflurane on the rate of neutrophil apoptosis in vitro. Ir J Med Sci 170:41-44.

Wei H, Kang B, Wei W, Liang G, Meng Q C, Li Y, Eckenhoff R G (2005) Isoflurane and sevoflurane affect cell survival and BCL-2/BAX ratio differently. Brain Res 1037:139-147.

Wei H, Liang G, Yang H, Wang Q, Hawkins B, Madesh M, Wang S, Eckenhoff R G (2008) The common inhalational anesthetic isoflurane induces apoptosis via activation of inositol 1,4,5-trisphosphate receptors. Anesthesiology 108:251-260.

Westphalen R I, Hemmings Jr H C (2006) Volatile anesthetic effects on glutamate versus GABA release from isolated rat cortical nerve terminals: basal release. J Pharmacol Exp Ther 316:208-215.

Wise-Faberowski L, Raizada M K, Sumners C (2001) Oxygen and glucose deprivation-induced neuronal apoptosis is attenuated by halothane and isoflurane. Anesth Analg 93:1281-1287.

Wise-Faberowski L, Aono M, Pearlstein R D, Warner D S (2004) Apoptosis is not enhanced in primary mixed neuronal/glial cultures protected by isoflurane against N-methyl-D-aspartate excitotoxicity. Anesth Analg 99:1708-1714.

Wise-Faberowski L, Zhang H, Ing R, Pearlstein R D, Warner D S (2005) Isoflurane-induced neuronal degeneration: an evaluation in organotypic hippocampal slice cultures. Anesth Analg 101:651-657.

Xie Z, Romano D M, Tanzi R E (2005a) Effects of RNAi-mediated silencing of PEN-2, APH-1a, and nicastrin on wild-type vs FAD mutant forms of presenilin 1. J Mol Neurosci 25:67-77.

Xie Z, Romano D M, Tanzi R E (2005b) RNA interference-mediated silencing of X11alpha and X11beta attenuates amyloid beta-protein levels via differential effects on beta-amyloid precursor protein processing. J Biol Chem 280:15413-15421.

Xie Z, Dong Y, Maeda U, Alfille P, Culley D J, Crosby G, Tanzi R E (2006a) The common inhalation anesthetic isoflurane induces apoptosis and increases amyloid beta protein levels. Anesthesiology 104:988-994.

Xie Z, Dong Y, Maeda U, Moir R, Inouye S K, Culley D J, Crosby G, Tanzi RE (2006b) Isoflurane-induced apoptosis: a potential pathogenic link between delirium and dementia. J Gerontol A Biol Sci Med Sci61:1300-1306.

Xie Z, Dong Y, Maeda U, Moir R D, Xia W, Culley D J, Crosby G, Tanzi R E (2007) The inhalation anesthetic isoflurane induces a vicious cycle of apoptosis and amyloid beta-protein accumulation. J Neurosci27:1247-1254.

Yang J, Zorumski C F (1991) Effects of isoflurane on N-methyl-D-aspartate gated ion channels in cultured rat hippocampal neurons. Ann NY Acad Sci 625:287-289.

Yon J H, Daniel-Johnson J, Carter L B, Jevtovic-Todorovic V (2005) Anesthesia induces neuronal cell death in the developing rat brain via the intrinsic and extrinsic apoptotic pathways. Neuroscience 135:815-827.

Zaugg M, Jamali N Z, Lucchinetti E, Shafiq S A, Siddiqui M A (2000) Norepinephrine-induced apoptosis is inhibited in adult rat ventricular myocytes exposed to volatile anesthetics. Anesthesiology 93:209-218.
Zhan X, Fahlman C S, Bickler P E (2006) Isoflurane neuroprotection in rat hippocampal slices decreases with aging: changes in intracellular Ca2+ regulation and N-methyl-D-aspartate receptor-mediated Ca2+ influx. Anesthesiology 104:995-1003.

Zhang S L, Yeromin A V, Zhang X H, Yu Y, Safrina O, Penna A, Roos J, Stauderman K A, Cahalan M D (2006) Genome-wide RNAi screen of Ca(2+) influx identifies genes that regulate Ca(2+) release-activated Ca(2+) channel activity. Proc Natl Acad Sci USA 103:9357-9362.

Zhao M, Su J, Head E, Cotman C W (2003) Accumulation of caspase cleaved amyloid precursor protein represents an early neurodegenerative event in aging and in Alzheimer's Disease. Neurobiol Dis 14:391-403.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

All definitions, as defined and used herein, should be understood to control over dictionary definitions, definitions in documents incorporated by reference, and/or ordinary meanings of the defined terms.

The indefinite articles “a” and “an”, as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of”, when used in the claims, shall have its ordinary meaning as used in the field of patent law.

It should also be understood that, unless clearly indicated to the contrary, in any methods claimed herein that include more than one act, the order of the acts of the method is not necessarily limited to the order in which the acts of the method are recited.

Each of the foregoing patents, patent applications and references that are recited in this application are herein incorporated in their entirety by reference, particularly for the teaching referenced herein.

We claim:

1. A method, comprising
administering an uncompetitive N-methyl D-aspartate (NMDA) receptor antagonist to a subject in temporal proximity to administering a halogenated ether anesthetic to the subject.

2. The method of claim 1, wherein the halogenated ether anesthetic is selected from the group consisting of isoflurane, enflurane, halothane, sevoflurane and desflurane.

3. The method of claim 2, wherein the halogenated ether anesthetic is isoflurane.

4. The method of claim 1, wherein the uncompetitive NMDA receptor antagonist is memantine or a pharmaceutically acceptable salt thereof.

5. The method of claim 1, wherein the uncompetitive N-methyl D-aspartate (NMDA) receptor antagonist and the halogenated ether anesthetic are administered to the subject within one hour of each other.

6. The method of claim 5, wherein the uncompetitive N-methyl D-aspartate (NMDA) receptor antagonist is administered to the subject prior to the halogenated ether anesthetic.

7. The method of claim 1, wherein the uncompetitive NMDA receptor antagonist is administered in an amount sufficient to decrease a level of amyloid-β or to prevent an increase in a level of amyloid-β in a cell of said subject and/or in an amount sufficient to reduce or prevent apoptosis in a cell in said subject and/or in an amount sufficient to prevent or inhibit a cognitive impairment or to decrease the level of a cognitive impairment in said subject.

8. The method of claim 1, wherein the subject is human.

9. A method for inhibiting postoperative cognitive dysfunction (POCD) induced by administration of a halogenated ether anesthetic to a subject comprising
administering an uncompetitive N-methyl D-aspartate (NMDA) receptor antagonist to a human subject in temporal proximity to administering the halogenated ether anesthetic to the subject.

10. The method of claim 9, wherein the halogenated ether anesthetic is selected from the group consisting of isoflurane, enflurane, halothane, sevoflurane and desflurane.

11. The method of claim 10, wherein the halogenated ether anesthetic is isoflurane.

12. The method of claim 1, wherein the uncompetitive NMDA receptor antagonist is memantine or a pharmaceutically acceptable salt thereof.

13. The method of claim 1, wherein the uncompetitive N-methyl D-aspartate (NMDA) receptor antagonist and the halogenated ether anesthetic are administered to the subject within one hour of each other.

14. The method of claim 13, wherein the uncompetitive N-methyl D-aspartate (NMDA) receptor antagonist is administered to the subject prior to the halogenated ether anesthetic.

15. The method of claim 1, wherein the uncompetitive NMDA receptor antagonist is administered in an amount sufficient to decrease a level of amyloid-β or to prevent an increase in a level of amyloid-β in a cell of said subject and/or in an amount sufficient to reduce or prevent apoptosis in a cell in said subject and/or in an amount sufficient to prevent or inhibit a cognitive impairment or to decrease the level of a cognitive impairment in said subject.

16. The method of claim 1, wherein the subject is human.

17. A method, comprising
administering an inhibitory nucleic acid molecule directed against IP3 receptor or SERCA1 and/or a chelating agent to a subject in temporal proximity to administering a halogenated ether anesthetic to the subject.

18. The method of claim 1, wherein the halogenated ether anesthetic is selected from the group consisting of isoflurane, enflurane, halothane, sevoflurane and desflurane.

19. The method of claim 1, wherein the inhibitory nucleic acid molecule and/or the chelating agent, and the halogenated ether anesthetic are administered to the subject within one hour of each other.

20. The method of claim 1, wherein the subject is human.


SEQUENCE LISTING (13 sequences)

SEQ ID NO: 1 - RNA, 21 nt, Homo sapiens
gccgggaucuuccugauuuuu

SEQ ID NO: 2 - RNA, 21 nt, Homo sapiens
  misc_feature (1)..(1): 5-phosphorylation
aaaucaggaagaucccggcuu

SEQ ID NO: 3 - RNA, 21 nt, Homo sapiens
ggagcacgcuggacucguuuu

SEQ ID NO: 4 - RNA, 21 nt, Homo sapiens
  misc_feature (1)..(1): 5′-phosphorylation
aacgaguccagcgugcuccuu

SEQ ID NO: 5 - RNA, 21 nt, Homo sapiens
gcaaucacauguggaaauuuu

SEQ ID NO: 6 - RNA, 21 nt, Homo sapiens
aauuuccacaugugauugcuu

SEQ ID NO: 7 - RNA, 21 nt, Homo sapiens
uggaaagucugaccgaauauu

SEQ ID NO: 8 - RNA, 21 nt, Homo sapiens
  misc_feature (1)..(1): 5′-phosphorylation
uauucggucagacuuuccauu

SEQ ID NO: 9 - RNA, 21 nt, Homo sapiens
ucgcacaaguccaagauuguu

SEQ ID NO: 10 - RNA, 21 nt, Homo sapiens
caaucuuggacuugugcgauu

SEQ ID NO: 11 - RNA, 21 nt, Homo sapiens
ggccaaaggugucuaugaguu

SEQ ID NO: 12 - RNA, 21 nt, Homo sapiens
cucauagacaccuuuggccuu

SEQ ID NO: 13 - RNA, 21 nt, Homo sapiens
uagcgacuaaacacaucaauu